(19) United States
(12) Patent Application Publication
(10) Pub. No.: US 2021/0267435 A1
(43) Pub. Date: Sep. 2, 2021

(54) A SYSTEM, METHOD AND COMPUTER PROGRAM FOR VERIFYING FEATURES OF A SCENE

(71) Applicant: Sony Corporation, Tokyo (JP)

(72) Inventors: Christopher WRIGHT, London (GB); Naoyuki HIROTA, Tokyo (JP)

(73) Assignee: Sony Corporation, Tokyo (JP)

(21) Appl. No.: 17/258,453

(22) PCT Filed: Oct. 9, 2019
(86) PCT No.: PCT/JP2019/039883

§ 371 Date: Jan. 7, 2021

(30) Foreign Application Priority Data
Oct. 12, 2018 (EP) 18200264.2

(51) Int. Cl. A61B 1/00; A61B 90/00

(52) U.S. Cl. CPC A61B 1/00057; A61B 2090/368; A61B 90/361; A61B 1/00009

(57) ABSTRACT

A verification system for verifying features of a scene, the system including circuitry configured to receive initial information determined in accordance with a first analysis of the scene, produce at least one test image in accordance with test information indicating at least one feature of the scene to be verified, the at least one test image being at least one predetermined image selected in accordance with the test information, modified in accordance with the initial information, overlay the scene with the at least one test image, receive comparison information relating to a comparison of the at least one test image overlaid with the scene with the at least one predetermined image selected in accordance with the test information and generate a verification status of a feature of the scene in accordance with the received comparison information.

### TECHNICAL FIELD

The present disclosure relates to a system, method and computer program for verifying features of a scene.

### BACKGROUND

The “background” description provided herein is for the purpose of generally presenting the context of the disclosure. Work of the presently named inventors, to the extent it is described in the background section, as well as aspects of the description which may not otherwise qualify as prior art at the time of filing, are neither expressly or impliedly admitted as prior art against the present disclosure.

In recent years, the technology and methods used for machine vision systems have undergone significant development, enabling robots and other computer systems to gain a detailed understanding of their surroundings based on visual input. As such, machine vision systems and automatic image analysis now plays an important role in the operation of many electronic and robotic devices. For example, machine vision is used in barcode reading, text translation, autonomous vehicle navigation, robotic surgical systems and the like. The information which is extracted from the image, and the complexity of the machine vision system, depends upon the particular application of the technology.

However, despite these recent advances, certain aspects of the technology and methods used in these systems are still under development. In fact, a number of problems arising from the use of machine vision systems have been reported. Failure of machine vision systems can occur due to previously undetected faults in the software or hardware used in such a system. These failures can, for example, result in machine vision systems misidentifying objects which a human operator would find simple to identify.

Machine vision systems can also be misled by conflicting inputs, adversarial images or the like. Adversarial images, caused by small changes in an input image, may trick the system into believing that an image of one item is actually an image of something else. These small changes may arise due to genuine fluctuations in the image feed, or may arise from a fraudulent attempt to mislead the system. Furthermore, many machine vision systems require precise initial calibration, and any mistake in this initial calibration could propagate throughout the system.

Often failures of the machine vision system will go unnoticed until such failures lead to malfunction of the robotic devices which rely upon the machine system.

The consequences of failures of machine vision systems vary significantly depending on the robotic systems and devices which rely upon that machine vision system. However, in recent years, machine vision systems have been used in increasingly complex situations, and a greater degree of reliance has been placed upon them. For example, in applications such as fault detection systems, vehicle navigation systems, surgical systems, collision avoidance systems and the like the consequences of failures of machine vision systems can be particularly severe.

Furthermore, the inherent complexity of machine vision systems leads to a lack of transparency in the working of machine vision systems to the end users. As such, many people continue to mistrust systems that rely on machine vision, even if the mistrust is misplaced. For example, surgeons are often reluctant to rely upon machine vision technology in robotic surgical devices owing to a lack of transparency in the machine vision system and genuine misunderstanding of the technology. Accordingly, there is resistance to the further implementation of machine vision technology, even in situations where such technology would provide significant advantages to traditional means. This leads to an underutilization of robotic devices and their associated machine vision systems

It is an aim of the present disclosure to address theses issues.

### SUMMARY

According to embodiments of the disclosure, a verification system for verifying features of a scene is provided, the system including circuitry configured to receive initial information determined in accordance with a first analysis of the scene, produce at least one test image in accordance with test information indicating at least one feature of the scene to be verified, the at least one test image being at least one predetermined image selected in accordance with the test information, modified in accordance with the initial information, overlay the scene with the at least one test image, receive comparison information relating to a comparison of the at least one test image overlaid with the scene with the at least one predetermined image selected in accordance with the test information and generate a verification status of a feature of the scene in accordance with the received comparison information.

According to embodiments of the disclosure, a verification method of verifying features of a scene, the method including receiving initial information determined in accordance with a first analysis of the scene, producing at least one test image in accordance with test information indicating at least one feature of the scene to be verified, the at least one test image being at least one predetermined image selected in accordance with the test information, modified in accordance with the initial information, overlaying the scene with the at least one test image, receiving comparison information relating to a comparison of the at least one test image overlaid with the scene with the at least one predetermined image selected in accordance with the test information and generating a verification status of a feature of the scene in accordance with the received comparison information.

According to embodiments of the disclosure, a computer program product including instructions which, when the program is executed by a computer, cause the computer to carry out the method including receiving initial information determined in accordance with a first analysis of the scene, producing at least one test image in accordance with test information indicating at least one feature of the scene to be verified, the at least one test image being at least one predetermined image selected in accordance with the test information, modified in accordance with the initial information, overlaying the scene with the at least one test image, receiving comparison information relating to a comparison of the at least one test image overlaid with the scene with the at least one predetermined image selected in accordance with the test information and generating a verification status of a feature of the scene in accordance with the received comparison information.

According to embodiments of the disclosure, instances where the machine vision system has misidentified objects within the scene can be identified prior to the operation of the robotic device, leading to a reduction in errors in robotic devices controlled by machine vision systems. Moreover, levels of machine vision system understanding can be intuitively assessed leading to an increase in the levels of trust between human operators and robotic devices.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is a view depicting an example of a schematic configuration of an endoscopic surgery system to which the technology according to an embodiment of the present disclosure can be applied;

FIG. 2 is a block diagram depicting an example of a functional configuration of the camera head and the CCU depicted in FIG. 1;

FIG. 3 illustrates a block diagram of an apparatus for verifying features of a scene according to embodiments of the disclosure;

FIG. 4A illustrates an exemplary situation of feature verification according to embodiments of the disclosure;

FIG. 4B illustrates an example of the production of a test image for an exemplary situation according to embodiments of the disclosure;

FIG. 5 illustrates a method of verifying features of a scene according to embodiments of the disclosure;

FIG. 6 depicts an exemplary table of test information which may be accessed by an apparatus in accordance with embodiments of the disclosure;

FIG. 7 illustrates an exemplary situation of overlaying the scene with augmented reality glasses according to embodiments of the disclosure;

FIG. 8 illustrates a method of verifying features of a scene according to embodiments of the disclosure;

FIG. 9 illustrates a method of verifying features of a scene according to embodiments of the disclosure;

FIG. 10 illustrates a method of verifying features of a scene according to embodiments of the disclosure;

FIG. 11 illustrates a method of verifying features of a scene according to embodiments of the disclosure;

FIG. 12 illustrates an exemplary situation of the correction of a projection for the operator location according to embodiments of the disclosure.

### DESCRIPTION OF EMBODIMENTS

Referring now to the drawings, wherein like reference numerals designate identical or corresponding parts throughout the several views.

Application

<<Application>>

The technology according to an embodiment of the present disclosure can be applied to various products. For example, the technology according to an embodiment of the present disclosure may be applied to an endoscopic surgery system, surgical microscopy or medical imaging device or other kind of industrial endoscopy in, say pipe or tube laying or fault finding.

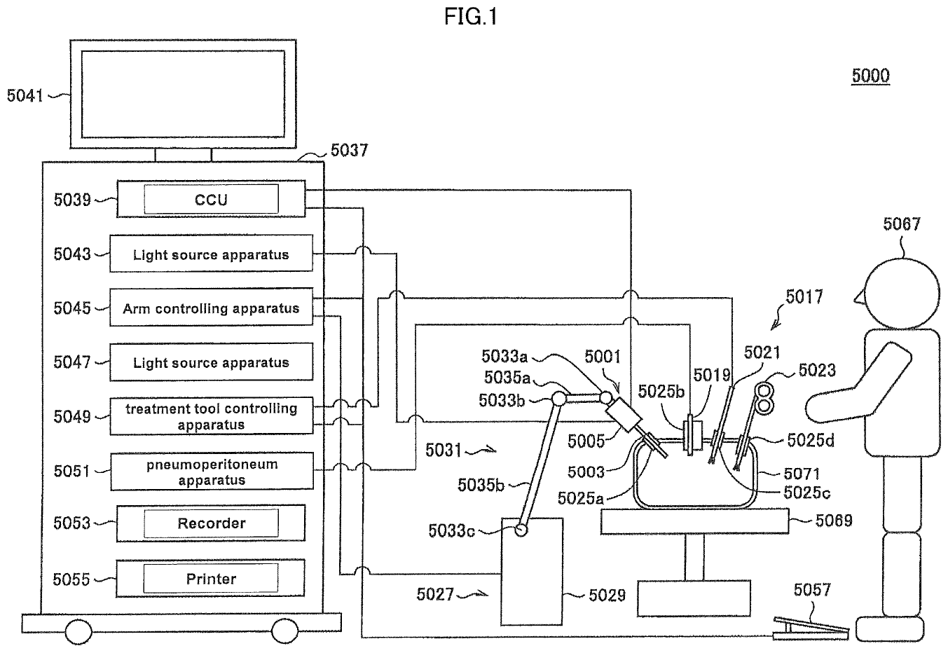

FIG. 1 is a view depicting an example of a schematic configuration of an endoscopic surgery system 5000 to which the technology according to an embodiment of the present disclosure can be applied. In FIG. 1, a state is illustrated in which a surgeon (medical doctor) 5067 is using the endoscopic surgery system 5000 to perform surgery for a patient 5071 on a patient bed 5069. As depicted, the endoscopic surgery system 5000 includes an endoscope 5001, other surgical tools 5017, a supporting arm apparatus 5027 which supports the endoscope 5001 thereon, and a cart 5037 on which various apparatus for endoscopic surgery are mounted.

In endoscopic surgery, in place of incision of the abdominal wall to perform laparotomy, a plurality of tubular aperture devices called trocars 5025a to 5025d are used to puncture the abdominal wall. Then, a lens barrel 5003 of the endoscope 5001 and the other surgical tools 5017 are inserted into body lumens of the patient 5071 through the trocars 5025a to 5025d. In the example depicted, as the other surgical tools 5017, a pneumoperitoneum tube 5019, an energy treatment tool 5021 and forceps 5023 are inserted into body lumens of the patient 5071. Further, the energy treatment tool 5021 is a treatment tool for performing incision and peeling of a tissue, sealing of a blood vessel or the like by high frequency current or ultrasonic vibration. However, the surgical tools 5017 depicted are mere examples at all, and as the surgical tools 5017, various surgical tools which are generally used in endoscopic surgery such as, for example, a pair of tweezers or a retractor may be used.

An image of a surgical region in a body lumen of the patient 5071 imaged by the endoscope 5001 is displayed on a display apparatus 5041. The surgeon 5067 would use the energy treatment tool 5021 or the forceps 5023 while watching the image of the surgical region displayed on the display apparatus 5041 on the real time basis to perform such treatment as, for example, resection of an affected area. It is to be noted that, though not depicted, the pneumoperitoneum tube 5019, the energy treatment tool 5021 and the forceps 5023 are supported by the surgeon 5067, an assistant or the like during surgery.

(Supporting Arm Apparatus)

The supporting arm apparatus 5027 includes an arm unit 5031 extending from a base unit 5029. In the example depicted, the arm unit 5031 includes joint portions 5033a, 5033b and 5033c and links 5035a and 5035b and is driven under the control of an arm controlling apparatus 5045. The endoscope 5001 is supported by the arm unit 5031 such that the position and the posture of the endoscope 5001 are controlled. Consequently, stable fixation in position of the endoscope 5001 can be implemented.

(Endoscope)

The endoscope 5001 includes the lens barrel 5003 which has a region of a predetermined length from a distal end thereof to be inserted into a body lumen of the patient 5071, and a camera head 5005 connected to a proximal end of the lens barrel 5003. In the example depicted, the endoscope 5001 is depicted which includes as a rigid type endoscope having the lens barrel 5003. However, the endoscope 5001 may otherwise be configured as a flexible type endoscope having the flexible type optical probe.

The lens barrel 5003 has, at a distal end thereof, an opening in which an objective lens is fitted. A light source apparatus 5043 is connected to the endoscope 5001 such that light generated by the light source apparatus 5043 is introduced to a distal end of the lens barrel by a light guide extending in the inside of the lens barrel 5003 and is irradiated toward an observation target in a body lumen of the patient 5071 through the objective lens. It is to be noted that the endoscope 5001 may be a forward viewing endoscope or may be a oblique viewing endoscope.

An optical system and an image pickup element are provided in the inside of the camera head 5005 such that reflected light (observation light) from an observation target is condensed on the image pickup element by the optical system. The observation light is photo-electrically converted by the image pickup element to generate an electric signal corresponding to the observation light, namely, an image signal corresponding to an observation image. The image signal is transmitted as RAW data to a CCU 5039. It is to be noted that the camera head 5005 has a function incorporated therein for suitably driving the optical system of the camera head 5005 to adjust the magnification and the focal distance.

It is to be noted that, in order to establish compatibility with, for example, a stereoscopic vision (three dimensional (3D) display), a plurality of image pickup elements may be provided on the camera head 5005. In this case, a plurality of relay optical systems are provided in the inside of the lens barrel 5003 in order to guide observation light to each of the plurality of image pickup elements.

(Various Apparatus Incorporated in Cart)

The CCU 5039 includes a central processing unit (CPU), a graphics processing unit (GPU) or the like and integrally controls operation of the endoscope 5001 and the display apparatus 5041. In particular, the CCU 5039 performs, for an image signal received from the camera head 5005, various image processes for displaying an image based on the image signal such as, for example, a development process (demosaic process). The CCU 5039 provides the image signal for which the image processes have been performed to the display apparatus 5041. Further, the CCU 5039 transmits a control signal to the camera head 5005 to control driving of the camera head 5005. The control signal may include information relating to an image pickup condition such as a magnification or a focal distance.

The display apparatus 5041 displays an image based on an image signal for which the image processes have been performed by the CCU 5039 under the control of the CCU 5039. If the endoscope 5001 is ready for imaging of a high resolution such as 4K (horizontal pixel number 3840×vertical pixel number 2160), 8K (horizontal pixel number 7680×vertical pixel number 4320) or the like and/or ready for 3D display, then a display apparatus by which corresponding display of the high resolution and/or 3D display are possible may be used as the display apparatus 5041. Where the apparatus is ready for imaging of a high resolution such as 4K or 8K, if the display apparatus used as the display apparatus 5041 has a size of equal to or not less than 55 inches, then a more immersive experience can be obtained. Further, a plurality of display apparatus 5041 having different resolutions and/or different sizes may be provided in accordance with purposes.

The light source apparatus 5043 includes a light source such as, for example, a light emitting diode (LED) and supplies irradiation light for imaging of a surgical region to the endoscope 5001.

The arm controlling apparatus 5045 includes a processor such as, for example, a CPU and operates in accordance with a predetermined program to control driving of the arm unit 5031 of the supporting arm apparatus 5027 in accordance with a predetermined controlling method.

An inputting apparatus 5047 is an input interface for the endoscopic surgery system 5000. A user can perform inputting of various kinds of information or instruction inputting to the endoscopic surgery system 5000 through the inputting apparatus 5047. For example, the user would input various kinds of information relating to surgery such as physical information of a patient, information regarding a surgical procedure of the surgery and so forth through the inputting apparatus 5047. Further, the user would input, for example, an instruction to drive the arm unit 5031, an instruction to change an image pickup condition (type of irradiation light, magnification, focal distance or the like) by the endoscope 5001, an instruction to drive the energy treatment tool 5021 or the like through the inputting apparatus 5047.

The type of the inputting apparatus 5047 is not limited and may be that of any one of various known inputting apparatus. As the inputting apparatus 5047, for example, a mouse, a keyboard, a touch panel, a switch, a foot switch 5057 and/or a lever or the like may be applied. Where a touch panel is used as the inputting apparatus 5047, it may be provided on the display face of the display apparatus 5041.

Otherwise, the inputting apparatus 5047 is a device to be mounted on a user such as, for example, a glasses type wearable device or a head mounted display (HMD), and various kinds of inputting are performed in response to a gesture or a line of sight of the user detected by any of the devices mentioned. Further, the inputting apparatus 5047 includes a camera which can detect a motion of a user, and various kinds of inputting are performed in response to a gesture or a line of sight of a user detected from a video imaged by the camera. Further, the inputting apparatus 5047 includes a microphone which can collect the voice of a user, and various kinds of inputting are performed by voice collected by the microphone. By configuring the inputting apparatus 5047 such that various kinds of information can be inputted in a contactless fashion in this manner, especially a user who belongs to a clean area (for example, the surgeon 5067) can operate an apparatus belonging to an unclean area in a contactless fashion. Further, since the user can operate an apparatus without releasing a possessed surgical tool from its hand, the convenience to the user is improved.

A treatment tool controlling apparatus 5049 controls driving of the energy treatment tool 5021 for cautery or incision of a tissue, sealing of a blood vessel or the like. A pneumoperitoneum apparatus 5051 feeds gas into a body lumen of the patient 5071 through the pneumoperitoneum tube 5019 to inflate the body lumen in order to secure the field of view of the endoscope 5001 and secure the working space for the surgeon. A recorder 5053 is an apparatus capable of recording various kinds of information relating to surgery. A printer 5055 is an apparatus capable of printing various kinds of information relating to surgery in various forms such as a text, an image or a graph.

In the following, especially a characteristic configuration of the endoscopic surgery system 5000 is described in more detail.

(Supporting Arm Apparatus)

The supporting arm apparatus 5027 includes the base unit 5029 serving as a base, and the arm unit 5031 extending from the base unit 5029. In the example depicted, the arm unit 5031 includes the plurality of joint portions 5033a, 5033b and 5033c and the plurality of links 5035a and 5035b connected to each other by the joint portion 5033b. In FIG. 1, for simplified illustration, the configuration of the arm unit 5031 is depicted in a simplified form. Actually, the shape, number and arrangement of the joint portions 5033a to 5033c and the links 5035a and 5035b and the direction and so forth of axes of rotation of the joint portions 5033a to 5033c can be set suitably such that the arm unit 5031 has a desired degree of freedom. For example, the arm unit 5031 may preferably be configured such that it has a degree of freedom equal to or not less than 6 degrees of freedom. This makes it possible to move the endoscope 5001 freely within the movable range of the arm unit 5031. Consequently, it becomes possible to insert the lens barrel 5003 of the endoscope 5001 from a desired direction into a body lumen of the patient 5071.

An actuator is provided in each of the joint portions 5033a to 5033c, and the joint portions 5033a to 5033c are configured such that they are rotatable around predetermined axes of rotation thereof by driving of the respective actuators. The driving of the actuators is controlled by the arm controlling apparatus 5045 to control the rotational angle of each of the joint portions 5033a to 5033c thereby to control driving of the arm unit 5031. Consequently, control of the position and the posture of the endoscope 5001 can be implemented. Thereupon, the arm controlling apparatus 5045 can control driving of the arm unit 5031 by various known controlling methods such as force control or position control.

For example, if the surgeon 5067 suitably performs operation inputting through the inputting apparatus 5047 (including the foot switch 5057), then driving of the arm unit 5031 may be controlled suitably by the arm controlling apparatus 5045 in response to the operation input to control the position and the posture of the endoscope 5001. After the endoscope 5001 at the distal end of the arm unit 5031 is moved from an arbitrary position to a different arbitrary position by the control just described, the endoscope 5001 can be supported fixedly at the position after the movement. It is to be noted that the arm unit 5031 may be operated in a master-slave fashion. In this case, the arm unit 5031 may be remotely controlled by the user through the inputting apparatus 5047 which is placed at a place remote from the surgery room.

Further, where force control is applied, the arm controlling apparatus 5045 may perform power-assisted control to drive the actuators of the joint portions 5033a to 5033c such that the arm unit 5031 may receive external force by the user and move smoothly following the external force. This makes it possible to move, when the user directly touches with and moves the arm unit 5031, the arm unit 5031 with comparatively weak force. Accordingly, it becomes possible for the user to move the endoscope 5001 more intuitively by a simpler and easier operation, and the convenience to the user can be improved.

Here, generally in endoscopic surgery, the endoscope 5001 is supported by a medical doctor called scopist. In contrast, where the supporting arm apparatus 5027 is used, the position of the endoscope 5001 can be fixed more certainly without hands, and therefore, an image of a surgical region can be obtained stably and surgery can be performed smoothly.

It is to be noted that the arm controlling apparatus 5045 may not necessarily be provided on the cart 5037. Further, the arm controlling apparatus 5045 may not necessarily be a single apparatus. For example, the arm controlling apparatus 5045 may be provided in each of the joint portions 5033a to 5033c of the arm unit 5031 of the supporting arm apparatus 5027 such that the plurality of arm controlling apparatus 5045 cooperate with each other to implement driving control of the arm unit 5031.

(Light Source Apparatus)

The light source apparatus 5043 supplies irradiation light upon imaging of a surgical region to the endoscope 5001. The light source apparatus 5043 includes a white light source which includes, for example, an LED, a laser light source or a combination of them. In this case, where a white light source includes a combination of red, green, and blue (RGB) laser light sources, since the output intensity and the output timing can be controlled with a high degree of accuracy for each colour (each wavelength), adjustment of the white balance of a picked up image can be performed by the light source apparatus 5043. Further, in this case, if laser beams from the respective RGB laser light sources are irradiated time-divisionally on an observation target and driving of the image pickup elements of the camera head 5005 is controlled in synchronism with the irradiation timings, then images individually corresponding to the R, G and B colours can be picked up time-divisionally. According to the method just described, a colour image can be obtained even if a colour filter is not provided for the image pickup element.

Further, driving of the light source apparatus 5043 may be controlled such that the intensity of light to be outputted is changed for each predetermined time. By controlling driving of the image pickup element of the camera head 5005 in synchronism with the timing of the change of the intensity of light to acquire images time-divisionally and synthesizing the images, an image of a high dynamic range free from underexposed blocked up shadows and overexposed highlights can be created.

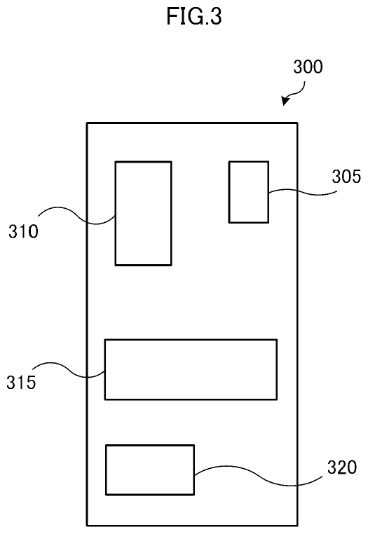

Further, the light source apparatus 5043 may be configured to supply light of a predetermined wavelength band ready for special light observation. This may include, but not be limited to laser light such as that provided by a Vertical Cavity surface laser or any kind of laser light. Alternatively or additionally, the light may be InfraRed (IR) light. In special light observation, for example, by utilizing the wavelength dependency of absorption of light in a body tissue to irradiate light of a narrower band in comparison with irradiation light upon ordinary observation (namely, white light), narrow band light observation (narrow band imaging) of imaging a predetermined tissue such as a blood vessel of a superficial portion of the mucous membrane or the like in a high contrast is performed. Alternatively, in special light observation, fluorescent observation for obtaining an image from fluorescent light generated by irradiation of excitation light may be performed. In fluorescent observation, it is possible to perform observation of fluorescent light from a body tissue by irradiating excitation light on the body tissue (autofluorescence observation) or to obtain a fluorescent light image by locally injecting a reagent such as indocyanine green (ICG) into a body tissue and irradiating excitation light corresponding to a fluorescent light wavelength of the reagent upon the body tissue. The light source apparatus 5043 can be configured to supply such narrow-band light and/or excitation light suitable for special light observation as described above. The light source may also apply a heat pattern to an area. This heat pattern will be explained later with reference to FIGS. 3A-C. The light source apparatus 5043 is, in embodiments, a Vertical Cavity Surface-Emitting Laser (VCSEL) which can produce light in the visible part of the electromagnetic spectrum and some produce light in the Infra-Red part of the electromagnetic spectrum. In this respect, the light source apparatus 5043 may also act as a visible light source illuminating the area. The light source apparatus 5043 is, in embodiments, one or more Vertical Cavity Surface-Emitting Laser (VCSEL) which can produce light in the visible part of the electromagnetic spectrum and some produce light in the Infra-Red part of the electromagnetic spectrum. In this respect, the light source apparatus 5043 may also act as a visible light source illuminating the area. The one or more VCSELs may be single wavelength narrowband VCSELs, where each VCSEL varies in emission spectral frequency. Alternatively, or additionally, one or more of the VCSELs may be a Micro Electro Mechanical system (MEMs) type VCSEL whose wavelength emission may be altered over a specific range. In embodiments of the disclosure, the wavelength may alter over the range 550 nm to 650 nm or 600 nm to 650 nm. The shape of the VCSEL may vary such as a square or circular shape and may be positioned at one or varying positions in the endoscope 5001.

The light source apparatus 5043 may illuminate one or more areas. This may be achieved by selectively switching the VCSELs on or by performing a raster scan of the area using a Micro Electro Mechanical system (MEMs). The purpose of the light source apparatus 5043 is to perform Spatial Light Modulation (SLM) on the light over the area. This will be explained in more detail later.

It should be noted that although the foregoing describes the light source apparatus 5043 as being positioned in the cart, the disclosure is not so limited. In particular, the light source apparatus may be positioned in the camera head 5005.

(Camera Head and CCU)

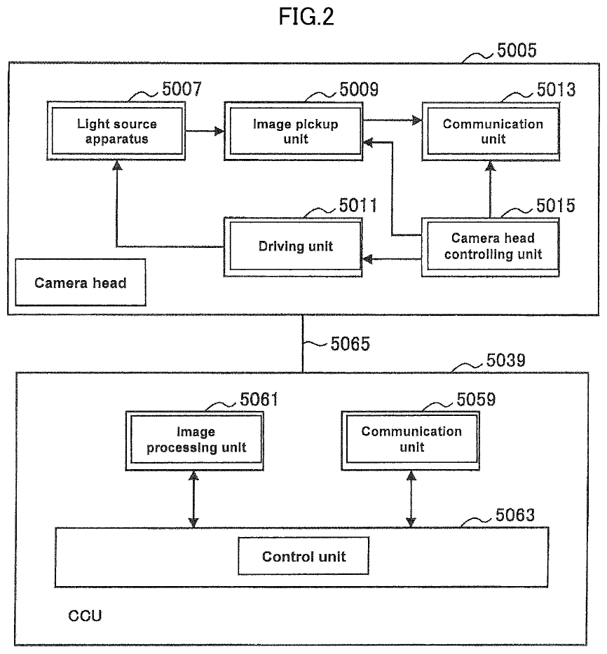

Functions of the camera head 5005 of the endoscope 5001 and the CCU 5039 are described in more detail with reference to FIG. 2. FIG. 2 is a block diagram depicting an example of a functional configuration of the camera head 5005 and the CCU 5039 depicted in FIG. 1.

Referring to FIG. 2, the camera head 5005 has, as functions thereof, a lens unit 5007, an image pickup unit 5009, a driving unit 5011, a communication unit 5013 and a camera head controlling unit 5015. Further, the CCU 5039 has, as functions thereof, a communication unit 5059, an image processing unit 5061 and a control unit 5063. The camera head 5005 and the CCU 5039 are connected to be bidirectionally communicable to each other by a transmission cable 5065.

First, a functional configuration of the camera head 5005 is described. The lens unit 5007 is an optical system provided at a connecting location of the camera head 5005 to the lens barrel 5003. Observation light taken in from a distal end of the lens barrel 5003 is introduced into the camera head 5005 and enters the lens unit 5007. The lens unit 5007 includes a combination of a plurality of lenses including a zoom lens and a focusing lens. The lens unit 5007 has optical properties adjusted such that the observation light is condensed on a light receiving face of the image pickup element of the image pickup unit 5009. Further, the zoom lens and the focusing lens are configured such that the positions thereof on their optical axis are movable for adjustment of the magnification and the focal point of a picked up image.

The image pickup unit 5009 includes an image pickup element and disposed at a succeeding stage to the lens unit 5007. Observation light having passed through the lens unit 5007 is condensed on the light receiving face of the image pickup element, and an image signal corresponding to the observation image is generated by photoelectric conversion of the image pickup element. The image signal generated by the image pickup unit 5009 is provided to the communication unit 5013.

As the image pickup element which is included by the image pickup unit 5009, an image sensor, for example, of the complementary metal oxide semiconductor (CMOS) type is used which has a Bayer array and is capable of picking up an image in colour. It is to be noted that, as the image pickup element, an image pickup element may be used which is ready, for example, for imaging of an image of a high resolution equal to or not less than 4K. If an image of a surgical region is obtained in a high resolution, then the surgeon 5067 can comprehend a state of the surgical region in enhanced details and can proceed with the surgery more smoothly.

Further, the image pickup element which is included by the image pickup unit 5009 includes such that it has a pair of image pickup elements for acquiring image signals for the right eye and the left eye compatible with 3D display. Where 3D display is applied, the surgeon 5067 can comprehend the depth of a living body tissue in the surgical region more accurately. It is to be noted that, if the image pickup unit 5009 is configured as that of the multi-plate type, then a plurality of systems of lens units 5007 are provided corresponding to the individual image pickup elements of the image pickup unit 5009.

The image pickup unit 5009 may not necessarily be provided on the camera head 5005. For example, the image pickup unit 5009 may be provided just behind the objective lens in the inside of the lens barrel 5003.

The driving unit 5011 includes an actuator and moves the zoom lens and the focusing lens of the lens unit 5007 by a predetermined distance along the optical axis under the control of the camera head controlling unit 5015. Consequently, the magnification and the focal point of a picked up image by the image pickup unit 5009 can be adjusted suitably.

The communication unit 5013 includes a communication apparatus for transmitting and receiving various kinds of information to and from the CCU 5039. The communication unit 5013 transmits an image signal acquired from the image pickup unit 5009 as RAW data to the CCU 5039 through the transmission cable 5065. Thereupon, in order to display a picked up image of a surgical region in low latency, preferably the image signal is transmitted by optical communication. This is because, upon surgery, the surgeon 5067 performs surgery while observing the state of an affected area through a picked up image, it is demanded for a moving image of the surgical region to be displayed on the real time basis as far as possible in order to achieve surgery with a higher degree of safety and certainty. Where optical communication is applied, a photoelectric conversion module for converting an electric signal into an optical signal is provided in the communication unit 5013. After the image signal is converted into an optical signal by the photoelectric conversion module, it is transmitted to the CCU 5039 through the transmission cable 5065.

Further, the communication unit 5013 receives a control signal for controlling driving of the camera head 5005 from the CCU 5039. The control signal includes information relating to image pickup conditions such as, for example, information that a frame rate of a picked up image is designated, information that an exposure value upon image picking up is designated and/or information that a magnification and a focal point of a picked up image are designated. The communication unit 5013 provides the received control signal to the camera head controlling unit 5015. It is to be noted that also the control signal from the CCU 5039 may be transmitted by optical communication. In this case, a photoelectric conversion module for converting an optical signal into an electric signal is provided in the communication unit 5013. After the control signal is converted into an electric signal by the photoelectric conversion module, it is provided to the camera head controlling unit 5015.

It is to be noted that the image pickup conditions such as the frame rate, exposure value, magnification or focal point are set automatically by the control unit 5063 of the CCU 5039 on the basis of an acquired image signal. In other words, an auto exposure (AE) function, an auto focus (AF) function and an auto white balance (AWB) function are incorporated in the endoscope 5001.

The camera head controlling unit 5015 controls driving of the camera head 5005 on the basis of a control signal from the CCU 5039 received through the communication unit 5013. For example, the camera head controlling unit 5015 controls driving of the image pickup element of the image pickup unit 5009 on the basis of information that a frame rate of a picked up image is designated and/or information that an exposure value upon image picking up is designated. Further, for example, the camera head controlling unit 5015 controls the driving unit 5011 to suitably move the zoom lens and the focus lens of the lens unit 5007 on the basis of information that a magnification and a focal point of a picked up image are designated. The camera head controlling unit 5015 may further include a function for storing information for identifying the lens barrel 5003 and/or the camera head 5005.

It is to be noted that, by disposing the components such as the lens unit 5007 and the image pickup unit 5009 in a sealed structure having high airtightness and waterproof, the camera head 5005 can be provided with resistance to an autoclave sterilization process.

Now, a functional configuration of the CCU 5039 is described. The communication unit 5059 includes a communication apparatus for transmitting and receiving various kinds of information to and from the camera head 5005. The communication unit 5059 receives an image signal transmitted thereto from the camera head 5005 through the transmission cable 5065. Thereupon, the image signal may be transmitted preferably by optical communication as described above. In this case, for the compatibility with optical communication, the communication unit 5059 includes a photoelectric conversion module for converting an optical signal into an electric signal. The communication unit 5059 provides the image signal after conversion into an electric signal to the image processing unit 5061.

Further, the communication unit 5059 transmits, to the camera head 5005, a control signal for controlling driving of the camera head 5005. The control signal may also be transmitted by optical communication.

The image processing unit 5061 performs various image processes for an image signal in the form of RAW data transmitted thereto from the camera head 5005. The image processes include various known signal processes such as, for example, a development process, an image quality improving process (a bandwidth enhancement process, a super-resolution process, a noise reduction (NR) process and/or an image stabilization process) and/or an enlargement process (electronic zooming process). Further, the image processing unit 5061 performs a detection process for an image signal in order to perform AE, AF and AWB.

The image processing unit 5061 includes a processor such as a CPU or a GPU, and when the processor operates in accordance with a predetermined program, the image processes and the detection process described above can be performed. It is to be noted that, where the image processing unit 5061 includes a plurality of GPUs, the image processing unit 5061 suitably divides information relating to an image signal such that image processes are performed in parallel by the plurality of GPUs.

The control unit 5063 performs various kinds of control relating to image picking up of a surgical region by the endoscope 5001 and display of the picked up image. For example, the control unit 5063 generates a control signal for controlling driving of the camera head 5005. Thereupon, if image pickup conditions are inputted by the user, then the control unit 5063 generates a control signal on the basis of the input by the user. Alternatively, where the endoscope 5001 has an AE function, an AF function and an AWB function incorporated therein, the control unit 5063 suitably calculates an optimum exposure value, focal distance and white balance in response to a result of a detection process by the image processing unit 5061 and generates a control signal.

Further, the control unit 5063 controls the display apparatus 5041 to display an image of a surgical region on the basis of an image signal for which image processes have been performed by the image processing unit 5061. Thereupon, the control unit 5063 recognizes various objects in the surgical region image using various image recognition technologies. For example, the control unit 5063 can recognize a surgical tool such as forceps, a particular living body region, bleeding, mist when the energy treatment tool 5021 is used and so forth by detecting the shape, colour and so forth of edges of the objects included in the surgical region image. The control unit 5063 causes, when it controls the display unit 5041 to display a surgical region image, various kinds of surgery supporting information to be displayed in an overlapping manner with an image of the surgical region using a result of the recognition. Where surgery supporting information is displayed in an overlapping manner and presented to the surgeon 5067, the surgeon 5067 can proceed with the surgery more safety and certainty.

The transmission cable 5065 which connects the camera head 5005 and the CCU 5039 to each other is an electric signal cable ready for communication of an electric signal, an optical fibre ready for optical communication or a composite cable ready for both of electrical and optical communication.

Here, while, in the example depicted, communication is performed by wired communication using the transmission cable 5065, the communication between the camera head 5005 and the CCU 5039 may be performed otherwise by wireless communication. Where the communication between the camera head 5005 and the CCU 5039 is performed by wireless communication, there is no necessity to lay the transmission cable 5065 in the surgery room. Therefore, such a situation that movement of medical staff in the surgery room is disturbed by the transmission cable 5065 can be eliminated.

An example of the endoscopic surgery system 5000 to which the technology according to an embodiment of the present disclosure can be applied has been described above. It is to be noted here that, although the endoscopic surgery system 5000 has been described as an example, the system to which the technology according to an embodiment of the present disclosure can be applied is not limited to the example. For example, the technology according to an embodiment of the present disclosure may be applied to a soft endoscopic system for inspection or a microscopic surgery system. Indeed, the technology may be applied to a surgical microscope for conducting neurosurgery or the like. Moreover, the technology may be applied more generally to any kind of medical imaging.

The technology according to an embodiment of the present disclosure can be applied suitably to the CCU 5039 from among the components described hereinabove. Specifically, the technology according to an embodiment of the present disclosure is applied to an endoscopy system, surgical microscopy or medical imaging. By applying the technology according to an embodiment of the present disclosure to these areas, blood flow in veins, arteries and capillaries may be identified. Further, objects may be identified and the material of those objects may be established. This reduces the risk to the patient's safety during operations.

The light source apparatus 5043 is, in embodiments, one or more Vertical Cavity Surface-Emitting Laser (VCSEL) which can produce light in the visible part of the electromagnetic spectrum and some produce light in the Infra-Red part of the electromagnetic spectrum. In this respect, the light source apparatus 5043 may also act as a visible light source illuminating the area. The one or more VCSELs may be single wavelength narrowband VCSELs, where each VCSEL varies in emission spectral frequency. Alternatively, or additionally, one or more of the VCSELs may be a Micro Electro Mechanical system (MEMs) type VCSEL whose wavelength emission may be altered over a specific range. In embodiments of the disclosure, the wavelength may alter over the range 550 nm to 650 nm or 600 nm to 650 nm. The shape of the VCSEL may vary such as a square or circular shape and may be positioned at one or varying positions in the endoscope system 5000.

The light source apparatus 5043 may illuminate one or more areas and/or objects within the areas. This may be achieved by selectively switching the VCSELs on or by performing a raster scan of the area using a Micro Electro Mechanical system (MEMs). The purpose of the light source apparatus 5043 is to perform Spatial Light Modulation (SLM) on the light over the area.

<Verifying Features of a Scene>

As noted above, it is desirable to verify features of a scene determined using machine vision systems or the like in order to reduce the instances of machine vision failure and to increase the level of trust end users have in the technology. As such, an apparatus for verifying features of a scene, which may be applied to a surgical scenario, is provided. According to embodiments of the disclosure, instances of machine vision failure can be significantly reduced.

By means of an example, machine vision systems (such as those used in a surgical scenario) may comprise one or more normal image sensors used to capture an image, and a subsequent image recognition processor used for detecting target objects in the captured image. In the surgical scenario, these target objects may comprise objects such as bones, blood vessels or a tumour. The machine vision system may also perform segmentation of the field of view of the captured image. The machine vision system may, alternatively or in addition to the normal image sensor, comprise sensing technology such as a NIR (near infrared) sensor for detecting fluorescence or for narrow band imaging, for example.

Furthermore, in order to obtain structural information, machine vision systems may comprise any type of 3D camera, such as stereoscopic cameras, depth sensors using structured light, time of flight information sensors, ultrasound technology, or the like.

FIG. 3 illustrates a block diagram of an apparatus for verifying features of a scene according to embodiments of the disclosure. The apparatus 300 includes a control device processor 305. The control device processor 305 is typically embodied as processor circuitry such as a microprocessor which is configured to operate using computer readable code. The control device processor 305 controls the operation of the device 300 using the computer readable code. Of course, the control device processor 305 may be embodied as hardware (such as an Application Specific Integrated Circuit or the like).

Additionally connected to the control device processor 305 is control device storage 310. The control device storage 310 is a computer readable storage medium (such as an optically readable, magnetically readable or solid state). The control device storage 310 is configured to store the computer readable code using which the control device processor 305 operates. In addition, user profiles and various data structures are stored in the control device storage 310.

Additionally connected to the control device processor 305 is control device communication circuitry 315. The control device communication circuitry 315 is configured to communicate with other devices which as may be required according to embodiments of the disclosure. This communication may be over a wired network (such as an Ethernet network) or may be over a wireless network (such as a WiFi network).

Finally, control device display circuitry 320 is connected to the control device processor 320. The control device display circuitry 320 is configured to display, to a user, test images overlaid upon a scene which have been produced in accordance with embodiments of the disclosure. Alternatively or additionally, the control device display circuitry 1220 may interact with an Augmented Reality (AR) system or a Virtual Reality (VR) system worn by a user, or may interact with an Augmented Reality projector system or the like as described with reference to embodiments of the disclosure.

Furthermore, the verification apparatus 300 may be provided as a system, with the control device processor 305, the control device communication circuitry 315, the control device display circuitry 320 and the control device storage 310 each being housed in a separate apparatus. The verification system may further comprise a display screen or projector, such as an augmented reality projector or the like, controlled by the control device display circuitry 320.

It will be appreciated that the apparatus for verifying features of the scene according to embodiments of the disclosure, described here with reference to FIG. 3, may be used in a surgical scenario such as that described with reference to FIG. 1 above. That is, the apparatus for verifying features of the scene 300 may be used with endoscopic surgery system 5000 for example.

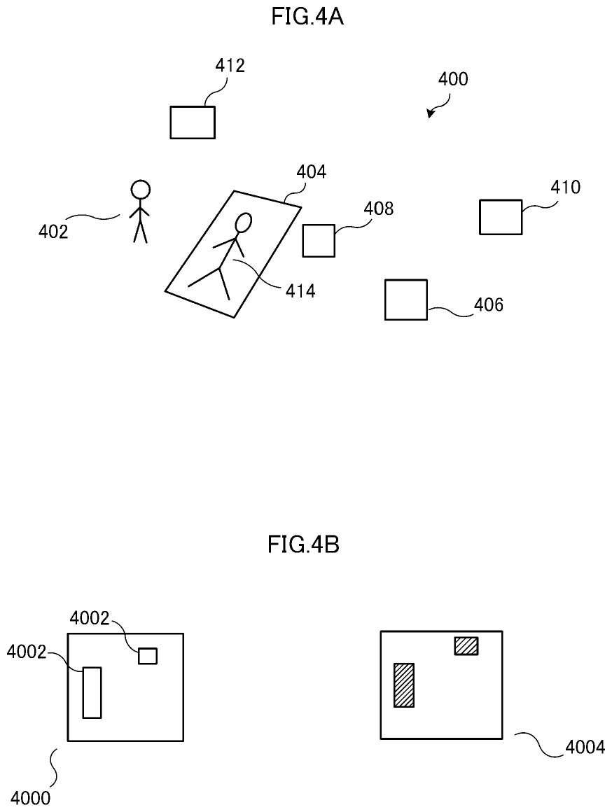

Furthermore, by means of an example, FIG. 4A illustrates an additional exemplary situation of feature verification according to embodiments of the disclosure. In this exemplary situation illustrated in FIG. 4A, surgeon 402 is present in a surgical theatre 400, the surgical theatre 400 further comprising an operating table 404, a machine vision system 406, a robotic apparatus 408 an apparatus for verifying features of the scene 410 (as described with reference to FIG. 3 above), a display device 412 and a patient 414 who is located on the operating table 404.

It will be appreciated that apparatus 410 can itself comprise a projector for projecting test images onto the scene or for projecting pointing guide to the surgeon on the scene. Furthermore, in the case that surgical procedure is performed by using surgical endoscope, this type of projection apparatus may be a micro projection device combined with the endoscope for projecting test image onto the scene.

In this exemplary situation, the surgeon 402 is performing an operation on the patient 414 alongside the robotic apparatus 408. That is, the robotic apparatus 408 is assisting the surgeon 402 in the operation, and may perform certain tasks autonomously on instruction of surgeon 402. Furthermore, the machine vision system 406 is connected to the robotic apparatus 408, and provides the robotic apparatus with information regarding the appropriate surgical site on or within patient 414.

In this exemplary situation, the machine vision system 406 is also connected to, or in communication with, the apparatus for verifying features of the scene 410. Finally, the apparatus for verifying features of the scene 410 is itself attached to, or in communication with, the display 412, and this display can be viewed by the surgeon 402.

The surgeon 402 is about to perform surgery to repair a fractured bone of patient 414 with the assistance of robotic device 408. Accordingly, machine vision system 406 views an image of the scene (in this case the operating table 404, a patient 414 on the operating table or part thereof) and extracts initial information of the scene from the image. Before the surgeon 402 begins the surgery, or before the surgeon assigns tasks to the robotic apparatus 408, the surgeon 402 wishes to verify that the machine vision system 406 connected to robotic apparatus 408 has correctly analysed the surgical site. That is, the surgeon 402 wishes to verify that the initial information extracted from the image by the machine vision system 406 have been correctly determined. Surgeon 402 therefore instructs apparatus 410 to verify the features of the scene determined by the machine vision system 406. That is, in this exemplary situation, the surgeon instructs the apparatus 410 that surgery to repair a fractured bone is going to be performed on patient 414 and requests that the apparatus 410 verifies the features of the surgical site determined by the machine vision system accordingly.

The apparatus for verifying features of the scene 410 receives the initial information determined by the machine vision system 406. The apparatus 410 may then obtain test information from a storage unit or local database. The test information indicates at least one feature of the scene which requires verification, and is selected in accordance with the information regarding the operation to be performed. That is, in this exemplary situation, since the operation relates to an operation to repair a fractured bone, the test information may indicate that the identification of the bone or bone fragments within the image by the machine vision system 406 must be verified.

Once the test information has been retrieved, the apparatus 410 produces a test image which will be used in order to verify the machine vision system has correctly identified the bone or bone fragments within the image. The test image is produced based upon a predetermined image identified by the test information, modified in accordance with the initial information received from the machine vision system 406. In this exemplary situation, the test information indicates that the test image should be based upon a direct image feed of the scene.

FIG. 4B illustrates an example of the production of a test image for an exemplary situation according to embodiments of the disclosure. In this example, the apparatus 410 modifies the direct image feed of the scene 4000 in accordance with the location of the bone or bone fragments 4002 determined in the initial information determined by the machine vision system 406. That is, in this exemplary situation, the apparatus 410 highlights the regions of the direct image feed 4000 where the machine vision system 406 has determined the bone or bone fragments 4002 to be located by changing the colours of the pixels in these regions. This modified image 4004 of the direct image feed 4000 is the test image which has been produced by the apparatus 410 in this exemplary situation.

Apparatus 408 is then further configured to overlay the test image 4004 with the direct image feed 4000 on the display device 412. That is, the apparatus 408 is configured to display the test image 4004 overlaid on the direct image feed 4000 of the scene on the display device 412. The apparatus 408 may also display an unedited view of the direct image feed 4000 on the display device 412 adjacent to the test image 4004 overlaid on the direct image feed 4000 for comparison.

Accordingly, the surgeon 402 can view the display device 412 in order to compare the test image 4004 overlaid with the scene and the predetermined image 4000 (the direct image feed of the scene). In this exemplary situation it is apparent to the surgeon that the correct regions of the image, corresponding to the locations of the bone and bone fragments 4002, have been highlighted by the apparatus 410. Since the correct region of the image has been highlighted by the apparatus 410 then the surgeon 302 can provide comparison information to the apparatus 410 confirming that this is the case. In contrast, if the test image 4004 produced by the apparatus 410 in accordance with the initial information received from the machine vision system 406 had highlighted an incorrect region of the image, the surgeon would realise that the bone fragments had not been highlighted and would inform the apparatus 410 accordingly.

Once the surgeon 402 has completed the comparison of the images on the display device 412, and has provided the apparatus 410 with comparison information, the apparatus uses this comparison information provided by the surgeon 402 in order to generate a verification status of the features in the scene. That is, in this exemplary situation, the apparatus 410 uses the comparison information in order to verify whether the features of surgical site have been correctly extracted from the original image of the surgical site by the machine vision system 406. In this case, since the bone fragments 4002 have been correctly highlighted, the apparatus 410 generates a verification status indicating that the initial image analysis has been correctly determined, and provides this information to the machine vision system 406 and/or robot apparatus 408.

The surgeon 402 may then proceed to perform the surgery to repair the fractured bone with confidence that the machine vision system 406 of the robotic apparatus 408 has correctly analysed the features of the surgical site.

In this manner, instances where the machine vision system has misidentified objects within the scene can be identified prior to the operation of the robotic device. Furthermore, apparatus 410 enables the surgeon to intuitively inspect the initial information provided by the machine vision system, leading to an increase in the level of trust between the surgeon 402 and the robotic device 408. Accordingly, resistance to the further implementation of machine vision technology can be reduced.

<Method for Feature Verification>

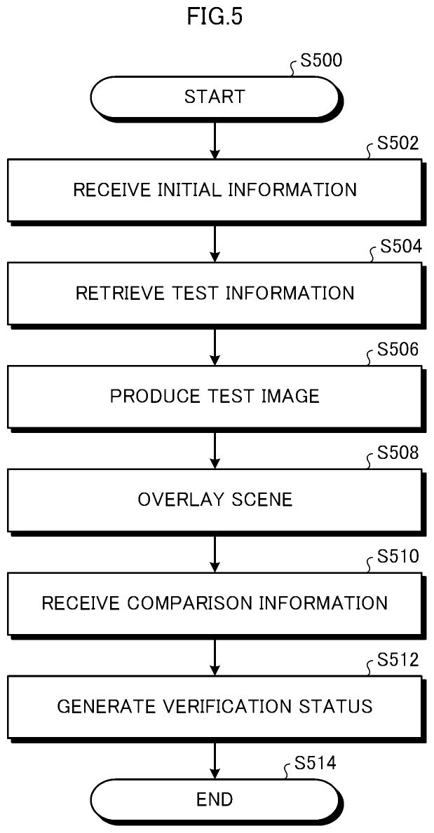

FIG. 5 illustrates a method of verifying features of a scene according to embodiments of the disclosure. Step S502 comprises receiving initial information determined in accordance with a first analysis of the scene. Step S504 comprises producing at least one test image in accordance with test information indicating at least one feature of the scene to be verified, the at least one test image being at least one predetermined image selected in accordance with the feature of the scene to be verified, modified in accordance with the initial information. Step S506 comprises overlaying the scene with the at least one test image. Step S508 comprises receiving comparison information relating to a comparison of the at least one test image overlaid with the scene with the at least one predetermined image selected in accordance with the test information. Finally, Step S510 comprises generating a verification status of a feature of the scene in accordance with the received comparison information.

It will be appreciated that the method according to the present embodiment may be performed on an apparatus (or alternatively a system or a server) as described with reference to FIG. 3. As previously stated, this apparatus 300 is controlled using a microprocessor or other processing circuitry 310. The apparatus is connected to the network and is able to receive transaction information from each node of the network. Apparatus 300 performs the method steps S502 to S510 described above with reference to FIG. 4A and FIG. 4B enabling features of a scene to be verified in accordance with embodiments of the disclosure, thus resulting a reduction in instances of machine vision failure.

Features of the operation of the apparatus 300 according to the embodiments of the disclosure are described in more detail below.

<Initial Information>

According to embodiments of the disclosure, the apparatus is configured to receive initial information determined in accordance with a first analysis of the scene. This information may include, for example, results of anatomical target detection, anatomical object recognition or result of segmentation of the scene (blood area, bone area, tumour or position of surgical tools or the like).

The initial information determined in accordance with the first image analysis of the scene corresponds to features which have been extracted by a machine vision system, such machine vision system 406 or the like, from an image of a scene. According to embodiments of the disclosure, the initial information includes detection or recognition information from a sensor information generated by a machine vision system. That is, the information received by the apparatus from a machine vision system or the like relates to an initial understanding of the features of the scene and may not, at that stage, have undergone any external verification. Of course, it will be appreciated that the method by which the initial information is produced is not particularly limited, and any such information regarding the features of the scene can be verified in accordance with the embodiments of the disclosure.

The types of features which have been extracted from the image of the scene by the machine vision system will vary considerably depending on the situation. Furthermore, the apparatus 300 according to embodiments of the disclosure may be configured to perform verification on all these features, or may only perform verification on a given subset of these features depending on test information. According to embodiments of the disclosure, the test information is retrieved in accordance with the operator information. The test information, and its retrieval using information supplied by the operator, is described in more detail below.

Furthermore, the mechanism by which the initial information is received by the apparatus 300 in accordance with embodiments of the disclosure is not particularly limited. That is, the information could be received over a wired network (such as an Ethernet network) or may be over a wireless network (such as a WiFi network). It will be appreciated that any such mechanism may be used for the reception of the initial information depending on the context of the situation to which embodiments of the disclosure are applied.

<Test Information>

According to certain embodiments of the disclosure, the apparatus may be configured to retrieve test information comprising information indicating at least one feature of the scene to be verified from a storage unit, in accordance with operator information. In other words, the apparatus 300 is configured to use information provided by the operator in order to retrieve information detailing which features of the scene are to be verified from a storage unit. Consider the exemplary situation described with reference to FIG. 4A above. In this exemplary situation, the surgeon 402 provides operator information describing the operation which is to be performed (such a surgery to repair a fractured bone or the like). The apparatus 410 uses this information to retrieve appropriate test information from a storage unit. The test information defines which features of the scene determined by the machine vision system need to be verified before the robotic device 408 is able to assist the surgeon 402 in that operation. In alternative embodiments, as described below, the test information may be selected in accordance with machine vision analysis of the scene. That is, for example, the machine vision system may identify a portion of the image which needs to be verified.

It will be appreciated that the operator information may be received by the apparatus 300 at any stage prior to the verification of the initial information. For example, the operator information could be provided to the apparatus as part of an initial set up, calibration or the like. Alternatively, the operator information could be provided to the apparatus when the robotic apparatus 408 is about to perform a new task. The operator information may be provided by any means, such as via a text input, a voice command, an input device, an input gesture or the like. Alternatively, the operator information could be provided remotely to the device over a communications network or the like. Of course, the form of the operator information itself is not particularly limited and can vary depending on the situation.

Once the apparatus has received the operator information, the apparatus is configured to retrieve test information from a storage unit. The test information, according to embodiments of the disclosure, relates to a predefined projection pattern for testing accuracy of the machine vision system which has been designed to enable the apparatus 300 to verify certain features of a scene. For example, the test information could instruct the apparatus 300 to highlight certain features on the surface of the scene. Failure of the apparatus 300 to do so indicates that the initial information provided by the machine vision system is inaccurate in this regard and should be recalibrated. The tests described by the test information may be of increasing difficulty and severity depending upon the varying accuracy requirements of the tasks and procedures which a robotic device relying upon the machine vision information will undertake. However, as stated above, the test information may also be selected by other means, such as in accordance with a machine vision analysis of the scene.

In other words, the test may be designed for specific applications, taking into account the known requirements of a machine vision system to successfully image the required features.

The test information may be stored locally in a storage unit contained in the apparatus or may, alternatively, be stored in an external database or the like. It will be appreciated that the test information is stored in the storage unit in a manner whereby it can readily be retrieved by the apparatus 300. For example, the test information may be stored in a manner whereby it can be extracted according to the function that test performs (colour check, feature recognition check, resolution check or the like); the level of complexity or accuracy of each test (such as the precision by which a feature will have to be identified in order to pass the test) or the specific tasks or procedures to which the test should be applied (relating to different types of surgery or operation for example). As such, the apparatus 300 is able to perform a search or lookup function in order to retrieve the most appropriate test information for a given situation.

For example, as described with reference to FIG. 4A, when the surgeon 402 indicates that the operation is related to surgery to repair fractured bones, then the test information which is retrieved indicates that features of the scene to be verified include the location of bones within the image.

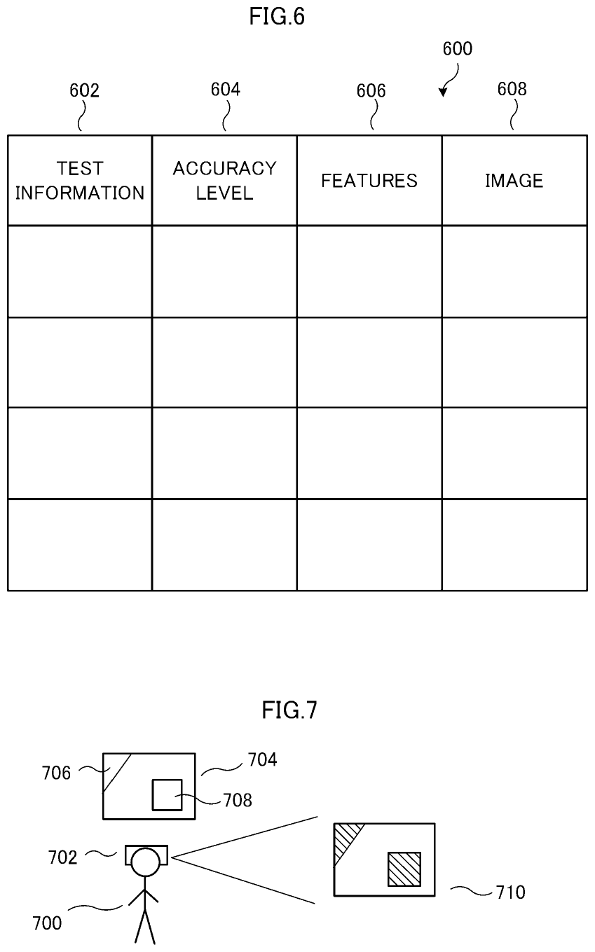

FIG. 6 depicts an exemplary table 600 of test information which may be accessed by the apparatus 300 in accordance with embodiments of the disclosure. In this exemplary table 600, each row corresponds to a separate test or set of test information 602. The columns of the table correspond to the different type of information contained in that test information 602. As depicted, such information may correspond to the required accuracy level 604, the features of the scene which are required to be verified 606, and the predetermined image 608 which is to be used in association with that test information 602. Specific examples of these predetermined images and the features of the image which they can be used to verify are described in more detail below. Of course, the information which is contained in the test information is not particularly limited in this regard and any such information may be included in accordance with embodiments of the disclosure as required.

It will be appreciated that the test information stored in the storage unit may be produced by various methods including being supplied by the operation robot manufacturer, being provided through an online platform, being created by the operator or the like. Furthermore, automatic test information may be produced using an external algorithm based, for example, upon the known capabilities of the machine vision system and the properties of the scene. 4

In certain exemplary situations, such as an initial calibration sequence during installation of a robotic device or a machine vision system in an operating space, the operator may be able to supply operator information to the apparatus 300 requesting that all the available relevant tests are performed sequentially by the apparatus 300 on a test surface or the like.

Furthermore, in certain exemplary situations, a robotic device may itself determine that one or more of the tests corresponding to the test information should be performed. That is, for example, depending on the surgery to be performed, the robotic device may decide which aspects of the machine vision system should be verified, and thus provide automated operator information to the apparatus 300 on this basis. Moreover, the automated operator information may be generated by the robotic device 300 in accordance with a confidence level provided by the machine vision system. That is, if the machine vision system has a low confidence level in the determination of object location for example, then the robotic device may provide the apparatus 300 with automated operator information requesting that test information which verifies the object location is used by the apparatus 300 for feature verification.

As such, according to certain embodiments of the disclosure, the test information describes a feature of the scene to be verified and a predetermined image which can be used, with the initial information, for the purposes of verifying that feature.

<Production of Test Image>

As described above, the apparatus 300 is configured to produce the at least one test image or test pattern which can be used for verifying features of the scene. The at least test image is a predetermined image selected in accordance with the feature of the scene to be verified, modified in accordance with the initial information. Furthermore, as described above, the feature of the scene to be verified is determined from the test information.

Consider the exemplary situation described with reference to FIG. 4A for example. In this exemplary situation, the test information indicates that the identification of bones and bone fragments in the surgical site by the machine vision system needs to be verified. In order to perform this verification, the test information has indicated that the predetermined image should be a direct image feed of the surgical site. This predetermined image is then modified in accordance with the initial information to highlight the regions of the image where the initial information (provided by the machine vision system) indicates that the bones or bone fragments are located. The scene is subsequently overlaid with the test image by the apparatus in order that a comparison between the test image overlaid with the scene and the predetermined image can be made.

Further exemplary methods of feature verification, and the production of the associated test image, which can be used in accordance with embodiments of the disclosure, are described in more detail below.

In certain embodiments, as described above, the test information may further indicate a required accuracy level of feature verification, and the apparatus 300 may be configured to produce the test image in accordance with this accuracy level requirement. That is, for example, the test information may indicate that bone fragments above a certain threshold size must be correctly identified by the machine vision system. In this situation, the test image would be created by the apparatus 300 in order to highlight those bone fragments in the image with sizes above the threshold limit. Alternatively, the test information may indicate that the location of the bones or bone fragments in the image must be determined to a certain degree of precision. In this case, the apparatus 300 may highlight regions of the image using a highlighter of a size corresponding to this required precision level. In this exemplary situation, provided the bones or bone fragments are located within these highlighted regions, then the location of the bones will be verified as being sufficiently determined by the machine vision system with regards to the required level of precision. Of course, the specific accuracy levels required may vary depending on the context of the situations to which embodiments of the disclosure are applied, and embodiments of the disclosure are not particularly limited in this regard.

In embodiments of the disclosure, the apparatus 300 is further configured to produce the test image in accordance with information regarding the operating environment. Details regrading the operating environment may be predetermined and provided to the apparatus 300 as initial calibration information for example. Alternatively or in addition, the apparatus 300 may be configured to determine information regarding the operating environment using additional sensors, camera systems or the like. Furthermore, information regarding the operating environment may be determined by an external device, such as the machine vision system, and subsequently provided to the apparatus 300.

For example, the apparatus 300 may produce the test image taking account of the amount of space available for projection of the test image onto the scene. Consider the exemplary situation illustrated in FIG. 4A for example. The apparatus 410 may produce the test image while taking account of the scale of the surgical site, in order that an appropriate size test image is produced for overlaying onto the scene. Of course, other environmental factors may be determined by the apparatus 300 and considered when producing the test image according to embodiments of the disclosure. For example, the apparatus 300 may produce the test image taking account of the ambient levels of light, in order to ensure that the projection of the test image can be seen by the human operator. Other environmental factors may be considered by the apparatus 300 when producing the test image depending on the context of the situation to which embodiments of the disclosure are applied.

Furthermore, in embodiments of the disclosure, the apparatus 300 is configured to produce the test image while taking account of the physical limitations of the display device on which the test image is to be displayed. For example, if the display device has a first resolution, then a test image which is to be overlaid on the scene using that display device should not be produced at a resolution exceeding the resolution of that display device. Otherwise, features of the test image may not be apparent to a person observing the display device (since the display device is unable to reproduce the test image at that resolution) and a person may, incorrectly, assume that the corresponding feature of the scene has been misunderstood by the machine vision system.

It will be appreciated that embodiments of the disclosure are not particularly limited in this regard, and other features of the display device may be considered by the apparatus 300 when producing the test image. For example, limitations on the colour depth of the display device or the like could be considered by the apparatus 300 when producing the test image for display.

The apparatus 300 may further be configured to consider the limitations of human vision when producing the test image. That is, when providing the comparison information, minor variations between the scene overlaid with the test image and the predetermined image may be unobservable to the human operator. Accordingly, the test image should be designed such that features are differentiated on a scale which will be perceivable to the human operator, in order that reliable comparison information can be obtained.

<Overlaying the Scene>

In embodiments of the disclosure, the apparatus 300 is configured to overlay feature of the scene with the at least one test image by displaying the at least one test image on a display. Of course, any suitable display device may be used in accordance with embodiments of the disclosure, depending on the context of the situation in which the embodiments of the disclosure are applied.

For example, in the exemplary situation described with reference to FIG. 4A, the scene has been overlaid with the test image on a display device 412 for comparison with the predetermined image. That is, the surgeon 402 views the display device 412 and makes a comparison between the image of the scene overlaid with the test image and the predetermined image. Once this comparison has been made, the surgeon 402 provides the apparatus 410 with the comparison information in order that a verification status can be generated by the apparatus 410 for the associated feature of the scene.

Alternatively, for example, the display device on which the images are displayed for comparison may be a head mounted display screen, such as augmented reality glasses or the like. Consider a further exemplary situation described with reference to FIG. 4A. In this exemplary situation, the apparatus 410 has produced the test image using the initial information received from the machine vision system 406, in accordance with the operator information received from the surgeon 402. The surgeon 402 is wearing augmented reality glasses, which enable the surgeon 402 to view the surgical site with additional information being added alongside the images of the scene. In this exemplary situation, the apparatus 410 communicates with the augmented reality glasses worn by the surgeon 402 in order that the test image is displayed by the augmented reality glasses such that the surgical site is overlaid with the test image.

FIG. 7 illustrates an exemplary situation of overlaying the scene with augmented reality glasses according to embodiments of the disclosure. In this example, the surgeon 700 is wearing the set of augmented reality glasses 702, and viewing the surgical site 704 through these glasses. The surgeon has instructed the apparatus 300 to verify the features of the scene 706 and 708. Apparatus 300 thus produces a test image highlighting the location of these features in the surgical site, and instructs the augmented reality glasses 702 to display the test image, such that when the surgeon 700 views the surgical site, they see the scene overlaid with the test image. The surgeon 700 thus sees image 710 when looking at the scene through the augmented reality glasses 702. In image 710, the surgeon 700 can see that the features of the scene have been correctly highlighted by the apparatus 300, providing the surgeon 700 with confidence that the machine vision system has correctly understood the features 706 and 708 of the scene 704. Furthermore, by displaying the test image on the augmented reality glasses 702 in this manner, the surgeon 700 can quickly and intuitively provide the comparison information to the apparatus 300 for feature verification without taking their eyes of the surgical site.

Alternatively, according to embodiments of the disclosure, the apparatus 300 is configured to overlay the scene with the at least one test image by projecting the at least one test image onto the scene. The projection of the test image onto the scene in this manner could be performed by an augmented reality projection system or the like. That is, the test image produced by the apparatus 300 could be projected directly onto the scene, such that a person viewing the scene would see the scene overlaid with the test image.

Consider the exemplary situation described above with reference to FIG. 4A. In this exemplary situation, as described above, the apparatus 410 has produced the test image using the initial information provided by the machine vision system 406, in accordance with the operator information provided by the surgeon 402. However, in this exemplary situation, the surgeon 402 is not wearing a head mounted display such as augmented reality glasses or the like. Rather, an augmented reality projector is provided in the surgical theatre 400. The position of the augmented reality projector is not particularly limited, provided it is capable of projecting images onto the surgical scene. The apparatus 410 then controls the augmented reality projector or the like in order that the test image is projected directly onto the surgical site. Accordingly, the surgeon 402 viewing the surgical site, without any additional glasses or display, will see the scene overlaid with the test image produced by the apparatus 410. The surgeon 402 can then provide comparison information regarding the scene to the apparatus 410. By displaying the test image on the augmented reality projector in this manner, the surgeon 402 can quickly and intuitively provide the comparison information to the apparatus 410 for feature verification without taking their eyes of the surgical site.

According to embodiments of disclosure when the at least one test image comprises a plurality of test images, the apparatus 300 is further configured to overlay the scene with the at least one test image in a sequence, and is further configured to receive comparison information for each of these test images in turn. In other words, if there are a number of features to be verified and a test image is produced for each of these features, or alternatively, if a single feature is to be verified, yet this feature is to be verified using a number of test images, then, according to embodiments of the disclosure, these test images will be overlaid on the scene in sequence.

In this manner, it is possible for the observer to view each of the test images overlaid with the scene in turn, and can provide comparison information relating to a comparison of each test image with the corresponding predetermined image. For example, according to embodiments of the disclosure where the test image is projected on the scene, the apparatus 300 may first cause the first test image to be projected onto the scene. Then, only once the comparison information has been received for this first test image, the projection changes to the second test image. According to embodiments of the disclosure, the operator could provide an input requesting that the projection should return to a previous test image. In this case, the projection would show the previous test image again, and the operator would be able to update the comparison information that they have provided regarding that test image. Alternatively, the test image projection may automatically change after a predetermined time, in order that the apparatus 300 cycles through the entire set of test images which are to be projected. Then, when the operator provides comparison information for a given test image, that test image will be removed from the cycle. The cycle will thus continue until the operator has provided comparison information for all of the test images.

In embodiments of the disclosure, the apparatus 300 will wait until comparison information has been received for all the test images before generating a verification status of a feature of the scene. Alternatively, the apparatus 300 will produce the feature verification status individually for each feature once the comparison for the test images corresponding to that feature has been received.

<Comparison Information>

As described above, once the test image has been produced by the apparatus 300, the scene is overlaid with the test image in order that the operator can provide comparison information. That is, the operator views the scene overlaid with the test image, and compares this with a predetermined image. The apparatus 300 then uses the comparison information in order to produce a verification status of the associated feature, as described in more detail below.

Consider the exemplary situation described with reference to FIG. 4A. Once the surgeon 402 has compared the images displayed on the display device 412, the surgeon 402 can provide the apparatus 410 with comparison information regarding the images. For example, in this exemplary situation, where the machine vision system 406 identification of bone and bone fragments appears correct, the surgeon 402 can provide confirmation of this fact to the apparatus 410.

It will be appreciated that such comparison information can be provided to the apparatus 300 according to embodiments of the disclosure by any input means, such as an input gesture, an input device or verbal commands such as speech recognition or the like. Use by the apparatus 300 of speech recognition or the like for receiving the comparison information may be advantageous in certain situations, since it enables the human operator to provide the comparison information whilst using their hands to perform other tasks or to operate additional equipment. Considering the exemplary situation illustrated with reference to FIG. 4A for example. Here, use by the apparatus 410 of speech recognition to receive the comparison information enables the surgeon 402 to provide the comparison information without releasing the equipment that they are currently using.

Furthermore, it will be appreciated that the form of the comparison information is not particularly limited, and may depend upon the context of the situation to which embodiments of the disclosure are applied. For example, in certain circumstances, the comparison information may comprise a simple indication of whether a required feature has been correctly identified or not. However, in more complex situations, where a number of features are to be verified, the comparison information may indicate which features of the image have been correctly identified, alongside an indication of the features of the image which have not been satisfactorily identified. Alternatively or in addition, the comparison information may indicate varying degrees of satisfaction. That is, the comparison information could indicate that certain features have been identified to a high precision, while other features have been identified to a lower precision.

According to embodiments of the disclosure, the apparatus 300 may, on the basis of the test information, provide guidance to the human operator of the comparison information which is required in a given situation. For example, according to embodiments of the disclosure, the apparatus 300 is further configured to generate comparison questions in accordance with the test information. These comparison questions could be visually or verbally communicated to the human operator, and may vary depending upon the feature or features which are to be verified.

Consider the exemplary situation described with reference to FIG. 4A. In this exemplary situation, the apparatus 410 has overlaid the scene with the test image on the display device 412. At this stage, the apparatus 410 may provide guidance to the surgeon 402 as to the comparison information which needs to be provided. In the exemplary situation whereby the surgeon 402 is performing surgery to repair a fractured bone with the assistance of robotic device 408, the apparatus 410 may ask the surgeon 402 to confirm whether all the bones or bone fragments in the image have been highlighted in the overlay of the scene with the test image. Alternatively, the apparatus 410 may ask the surgeon 402 to identify whether any part of the surgical site has been highlighted which does not correspond to a bone or bone fragment. In this manner, the apparatus 410 guides the surgeon 402 to provide the comparison information required to generate a verification status for the features of the scene in accordance with the test information thus further reducing the instances of machine vision misidentification.

<Verification Status>

Once the apparatus 300 receives the comparison information, it uses the comparison information in order to generate a verification status of the feature of the scene. That is, the apparatus 300 is configured to generate a verification status of a feature of the scene in accordance with the received comparison information.

For example, in the exemplary situation of FIG. 4A, when the surgeon 402 indicates that the correct regions of the surgical site have been highlighted by the apparatus 410, the apparatus 410 will generate a verification status that verifies that the feature of bone location has been correctly identified by the machine vision system 406. Alternatively, when the surgeon 402 expresses a level of concern or dissatisfaction in the comparison information, the apparatus 410 will generate a verification status which indicates that the feature of bone location has not been correctly determined by the machine vision system 406.

It will be appreciated that the form of the verification status is not particularly limited, and may vary in accordance with the context of the situation to which embodiments of the disclosure are applied. For example, in some exemplary situations, the verification status generated by the apparatus 300 may be a binary signal indicating whether or not the feature has been correctly identified. In some exemplary situations, the apparatus 300 may produce a single verification status for the feature while, in other exemplary situations, the apparatus 300 may produce a plurality of verification status indications corresponding to different aspects of the feature which have been verified. In the situations whereby the test information indicates that a number of features are to be verified, the apparatus 300 may produce an individual verification status for each feature, or alternatively, may produce a single verification status for all features.

In some embodiments of the disclosure, the test information may indicate a required level of confidence which the human operator must express in a certain feature in order for that feature to be verified by the apparatus 300. The actual level of confidence of the human operator in that feature is determined from the comparison information provided by the human operator to the apparatus 300. Accordingly, certain features may require a high degree of confidence in order to be verified, while other features of lesser importance in the given situation may require only a low degree of confidence in order to be verified.

In some embodiments of the disclosure, the apparatus 300 may further be configured in order to use the verification status to provide a warning or indication to the human operator or robotic device not to continue with a procedure when the verification status indicates that the features have not been correctly determined by the machine vision system. Alternatively, the verification status may be used in order to generate a recalibration request, the recalibration request instructing the machine vision system to perform recalibration and to produce further information regarding the scene for use in a secondary verification attempt.

Furthermore, it will be appreciated that according to embodiments of the disclosure, the verification status may indicate which aspects of the initial information have been incorrectly determined when producing the verification status. In this manner, the apparatus 300 may instruct the machine vision system on which features of the machine vision system require recalibration.

<Exemplary Methods of Feature Verification>

In the exemplary situation described with reference to FIG. 4A, the apparatus 410 determined from the test information, selected on the basis of the operator information, that the features to be verified in the scene were the locations of certain objects, such as bone fragments, in that scene. However, it will be appreciated that embodiments of the disclosure are not particularly limited to object recognition verification. Rather, there are numerous examples of features of the scene which can be verified in accordance with embodiments of the disclosure. As described above, the specific features to be verified will depend upon the test information which is selected according to the operator information, and will therefore vary depending on the context of the situation to which embodiments of the disclosure applied.

Consider the exemplary situation described with reference to FIG. 4A above. Undetected variations in topology can affect the ability of the robotic device 408 depending on the machine vision system 406 to make accurate surgical actions. Therefore, verification of the machine vision system's understanding of surface features, such as roughness of the scene, may be important in certain situations, and should be verified before use.

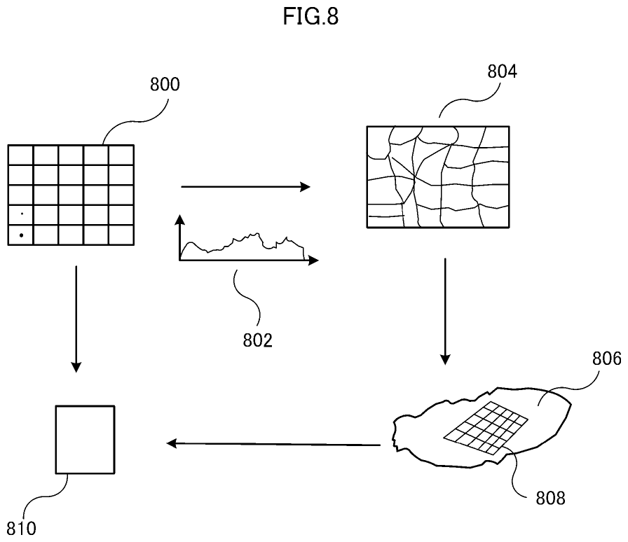

FIG. 8 illustrates a method of verifying features of a scene according to embodiments of the disclosure. In this exemplary situation, the feature to be verified is the feature of the surface topology. The predetermined image 800 is modified by the surface topology 802 which has been received in the initial information from the machine vision system to form the test image 804. The test image is then projected onto the scene 806 to form the overlay of the scene with the test image 808. A comparison is made between the projection of the test image 808 and the predetermined image 800 by the verification apparatus 810 (which corresponds to apparatus 300 described with reference to FIG. 3).

It will be appreciated that the exact form of the predetermined image 800 is not particularly limited. Rather, the predetermined image 800 merely provides an initial image which can be used by the apparatus 810 to test the topology of the scene. That is, any such predetermined image or projection pattern may be used in accordance with embodiments of the disclosure. The initial information received in by the apparatus 810 from the machine vision system provides an initial topology of the scene 802; it is this topology of the scene 802 which is to be verified according to the present example. In this case, topology is indicative of three dimensional information of the object. For example, shape of the surface of the object or depth information of captured image of the object captured by any types of three dimensional vision system such as stereoscopic image sensor, 3D sensor using structured light or ultrasound technology, or time-of-flight camera. As stated above, the method by which the machine vision system has determined this initial topology is not particularly limited. Once the predetermined image 800 has been retrieved by the apparatus 810, then the initial topology 802 is applied to the predetermined image 800 in order to create image 804. That is, the predetermined image is modified by the apparatus 810 using the initial information in a manner such that when projected onto a surface having a topology as illustrated by 802, then the projection of the test image 804 on the scene 806 will appear as an undistorted version of predetermined image 800.

In other words, the apparatus 810 distorts the image 800 based on the initial topology information 802 in order to produce test image 804 which will reproduce the initial image 800 only if the test image 804 is projected onto a surface having that initial topology 802. If the distorted image 804 is projected onto a surface which does not have the topology 802, then the projection 808 will not appear undistorted by the person viewing the projection. As such, if, following projection, the image is still distorted, then it can be determined that the machine vision understanding of the topological variation of the scene is flawed.

The comparison information provided by the operator in this situation could simply indicate that the topology has been incorrectly determined, or alternatively, could indicate specific regions of the topology which have been shown to be particularly problematic. Accordingly, upon generation of the verification status, the apparatus may indicate to the machine vision system aspects of the topology analysis which need to be recalculated.

It will be appreciated that this exemplary method of topology verification requires the test image to be projected directly onto the scene.

Subtle variations in colour and brightness across a scene can differentiate tissues and other features within the scene. If the machine vision system has incorrectly determined the colour and or brightness variation across an image then certain features of the scene may therefore be misidentified. Consider the exemplary situation depicted in FIGS. 4A and 4B for example. In this exemplary situation, features such as a bone or bone fragments may appear more white or brighter than the surrounding tissue. Accordingly, correctly determining the colours and brightness in the scene will improve the differentiation between bone and tissue in the scene.

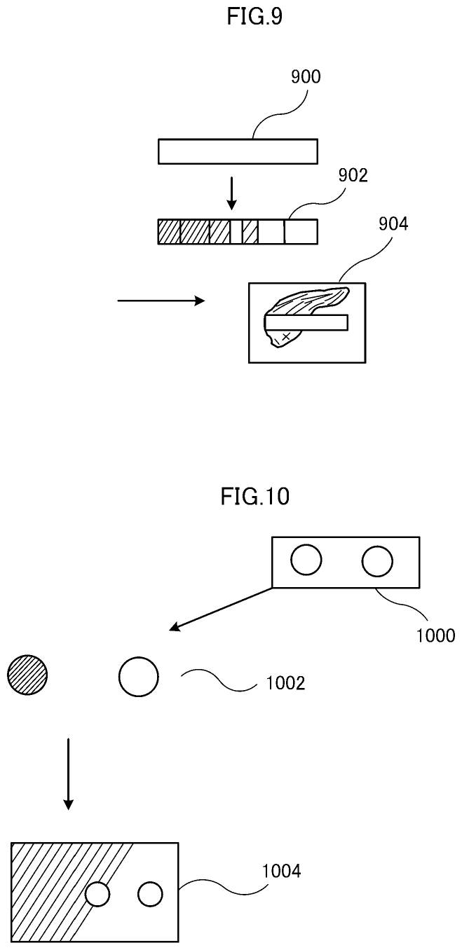

FIG. 9 illustrates a method of verifying features of a scene according to embodiments of the disclosure. In this exemplary situation, the feature to be verified is the understanding of colour and or brightness variations across the scene. According to embodiments of the disclosure, when the test information determined in accordance with the operator information indicates that the colour and or brightness variation across the scene should be verified, the apparatus 300 obtains a predetermined image 900 for use in production of the test image 902. It will be appreciated that embodiments of the disclosure are not particularly limited to the form of the predetermined image 900. However, in this example, image 900 should be an image of uniform colour and/or brightness.

Once the predetermined image 900 has been retrieved by the apparatus 300, the apparatus modifies predetermined image 900 in accordance with the initial information received from the machine vision system. That is, in the situation whereby the colour and/or brightness of the image is to be verified, then the method according to embodiments of the disclosure comprises varying the colour and/or brightness of the predetermined image in accordance with the initial information in order that a line of single uniform colour and/or brightness is produced when that modified image is projected directly onto the scene having that colour and/or brightness variation.

That is, if the line of uniform colour (such as that in predetermined image 900) is projected directly onto the scene, then a person who views the scene will not see a line of uniform colour and/or brightness. Rather, they will see a line where the colour and/or brightness varies across the scene, since the scene onto which the line is projected is not a scene of uniform colour. In contrast, if the machine vision system has correctly analysed the scene, and the test image 902 is distorted appropriately, then, when the test image 902 is projected onto the scene, a line of uniform colour will be visible to a user, since the apparatus 300 will have correctly compensated for the colour variation across the scene.

In other words, if the user observes the overlay of the scene with the test image and determines that the line is not a uniform colour and/or brightness then this is an indication that the machine vision system is not detecting the colour and/or brightness of the scene correctly. In this case, the apparatus 300 can generate a feature verification status that requests that the colour and/or brightness of the scene is recalibrated by the machine vision system. According to embodiments of the disclosure, the feature verification status could indicate that the colour and/or brightness variation across the entire scene has been determined unsatisfactorily. Alternatively or in addition, the feature verification status could indicate that the colour and/or brightness variation of specific regions of the scene need to be recalibrated by the machine vision system before the operation can proceed.

It will be appreciated that this exemplary method of colour and/or brightness verification requires the test image to be projected directly onto the scene.

Machine vision systems can find specular reflections, where light is reflect at the same angle to the surface normal as the incident ray, difficult to understand, owing to the fact that the reflective properties of the surface may vary considerably over small scale variations. In contrast to specular reflection, where the light is reflected at a single angle from the surface, diffuse reflections occur when light is scattered at many angles from the surface. Specular reflections will only be observed when the angle at which the reflection is viewed is the same as the angle of incidence of the light (measured from the surface normal).

The reflectivity of a scene will vary considerably according to the objects which are located with the scene. Consider the exemplary situation described with reference to FIG. 4A above. In this situation, reflectivity of certain types of tissue located in the surgical site may have considerably higher levels of reflectivity than other types of objects which may located in the surgical site. Accordingly, the reflectivity can be used to differentiate between these objects.

It may therefore be advantageous, in certain situations, to test the understanding of the machine vision system of the reflectivity of the surface. The situations in which this feature of the scene requires verification are detailed in the test information which is retrieved in accordance with the operator information.

FIG. 10 illustrates a method of verifying features of a scene according to embodiments of the disclosure. In this exemplary situation, the feature to be verified is the understanding of the reflectivity of objects across the scene. According to embodiments of the disclosure, when the test information retrieved using the operator information indicates that the understanding of reflectivity of the surface needs to be verified, the apparatus 300 obtains an associated predetermined image 1000. The predetermined image 1000 will be used with the initial information regarding the reflectivity of the surface received from the machine vision system in order to produce test image 1002 which is to be overlaid on the scene. In this exemplary method, the predetermined image 1000 is an image of two identical circles; these circles are circles of the same intensity. The information regarding the reflectivity of the surface is then used in order to produce a test image 1002 where the circles have a different intensity. That is, the apparatus is configured to modify the intensity of the circles such that when the test image is projected onto a surface having the reflectivity described in the initial information then the circles of the projected test image will appear to have the equal intensity.

Subsequently, the apparatus 300 is configured to project the test image onto the surface 1004. If the surface reflectivity in the initial information has been correctly determined by the machine vision system then the circles will appear to be of equal intensity to an observer viewing the projection of the test image on the surface 1004. However, if the circles in the projected image appear to have different intensity, then the user can provide this information to the apparatus 300 in the comparison information. The apparatus 300 will then generate the feature verification status, and may, according to embodiments of the disclosure, require that the machine vision system from which the initial information is received is recalibrated.

It will be appreciated that this exemplary method of reflectivity verification requires the test image to be projected directly onto the scene.

An appropriate understanding of the variation of translucence across the scene may be required by the machine vision system. That is, the operator may wish to verify that the machine vision system has correctly determined the variation of translucence across the scene. Consider the exemplary situation illustrated with respect to FIG. 4A. Certain objects in a surgical site may be considerably less translucent than other certain objects in that surgical site. For example, bones and bone fragments in the image will have a very low value of translucence since they are almost opaque to visible light. In contrast, other objects, such as organs or tissue, will have a considerably higher level of translucence.

Light projected onto translucent objects will appear blurred, owing to the multi-depth reflection from within the translucent material. That is, some of the incident light will be reflected off the surface of the translucent tissue, while other portions of the incident light will be reflected at varying depths from within the translucent tissue. In contrast, the majority of the light incident upon an almost opaque object will be reflected from the surface of that object.

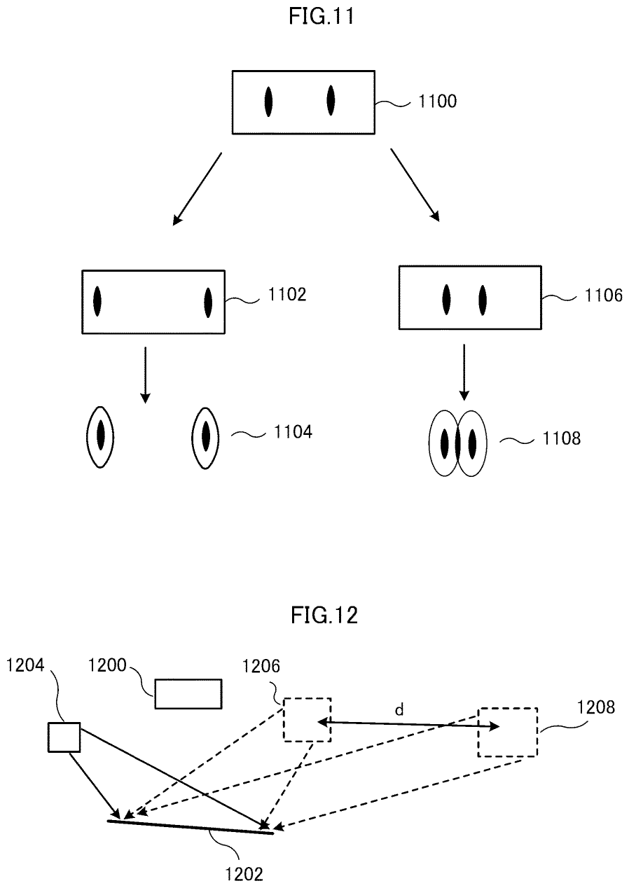

FIG. 11 illustrates a method of verifying features of a scene according to embodiments of the disclosure. Accordingly, once initial information regarding the translucence of tissue has been determined by the machine vision system and provided to the apparatus 300 in accordance with the embodiments of the disclosure, then an exemplary method such as that illustrated in FIG. 11 can be used to verify the machine vision systems understanding of the variation in translucence across the image.

According to embodiments of the disclosure, when the test information retrieved using the operator information indicates that the understanding of translucence needs to be verified, the apparatus 300 obtains an associated predetermined image 1100. The predetermined image will be used with the initial information regarding the translucence received from the machine vision system in order to produce test image 1102 or 1104 which is the test image to be overlaid on the scene. In this exemplary method, the predetermined image is an image of two identical lines. These lines, in the predetermined image, are set at a fixed distance away from each other.

From the initial information, indicating the translucence of the tissue which has been determined by the machine vision system, the apparatus 300 can determine the level of blurring which will occur when the two lines of the predetermined image 1100 are projected onto the scene. According to this exemplary method of verifying the translucence of the image, the apparatus 300 then modifies the predetermined image according to the initial information such that the lines are associated a second distance away from each other. That is, the apparatus 300 changes the distance between the lines in accordance with the initial information received from the machine vision system.

This modified predetermined image then forms the test image 1102 or 1104 which is to be projected onto the scene. The distance of separation is determined by the apparatus 300 to be the distance of separation between the two lines where, if the test image is projected onto a surface having the translucence as described in the initial information the amount of blurring of the lines will cause a small region of overlap between the blurred regions which will appear as a third line to a person observing the projection of the test image onto the surface.

If the translucence of the surface has been incorrectly determined by the machine vision system, then the distance of separation between the lines in the test image may be set at a distance which is too large 1102. In this case, when the test image is projected onto the surface, the person observing the projected image will not observe any overlap between the blurred regions 1104 and will realise that the translucence has been incorrectly determined by the machine vision system. The person can then provide this comparison information to the apparatus 300, which will generate the feature verification status accordingly.

Alternatively, consider the case whereby the translucence has been correctly determined by the machine vision system, and the lines in the test image 1106 are set at the correct distance apart. In this case, when the test image 1106 is projected onto the surface, the person observing the projected image will observe a region of overlap between the blurred regions 1108, and will realise that the translucence has been correctly determined by the machine vision system. The person can then provide this comparison information to the apparatus 300, which will generate the feature verification status accordingly.

Finally, if the translucence has been incorrectly determined, the lines may be set too close together in the test image. In this case, when the test image is projected onto the surface, the person would observe a region of overlap which is too large, and will realise that the translucence has been incorrectly determined by the machine vision system. The person can then provide this comparison information to the apparatus 300, which will generate the feature verification status accordingly.

Furthermore, comparison information regarding whether the lines are too far apart or too close together can provide important information as to the manner by which the translucence of the surface has been incorrectly determined. That is, if, when the test image is projected onto the surface, the lines appear too far apart, then it can be determined that less blurring than anticipated has occurred and thus that the actual translucence of the surface is lower than the translucence in the initial information. Alternatively, if, when the test image is projected onto the surface, the lines appear too close together, then it can be determined that more blurring than anticipated has occurred and thus that the actual translucence of the surface is higher than the translucence in the initial information. This additional information regarding the manner by which the translucence of the surface has been incorrectly determined by the machine vision system can be included in the feature verification status produced by the apparatus 300.

It will be appreciated that this manner of translucence verification requires projection of the test image onto the surface using an augmented reality projector or the like.

The above descriptions have been provided as exemplary methods by which features of the scene can be verified by the apparatus 300 according to embodiments of the disclosure. However, it will be appreciated that the present disclosure is not particularly limited in this regard, and other methods and features can be used and verified according to the context of the situation in which the embodiments of the disclosure are implemented.

<Location Information>

As described above, in certain embodiments of the disclosure, overlaying the scene with the test image comprises projecting the test image which has been produced by the apparatus 300 directly onto the scene using an augmented reality projector or the like. This enables the feature verification system to verify physical features of the scene such as the surface topology, colour variation, translucence and the like. However, it will be appreciated that certain aspects of the projection may vary depending from the location from which they are viewed by a user.

Accordingly, in certain embodiments, there may be a predetermined fixed or central location from which the user is required to view the projection in order to verify the features of the scene. Such a location may be calibrated upon initial setup of the apparatus 300 for example. It will be appreciated that the manner by which the predetermined location is communicated to the user is not particularly limited. For example, the predetermined location could be identified using the augmented reality projector or the like to highlight the viewing location on the floor. Alternatively, the predetermined location could be communicated to the operator or a display screen, or could be communicated through verbal instructions, such a simple direction description, provided to the user.

However, according to embodiments of the disclosure, the apparatus 300 may further be configured to detect a location of a person viewing the projection and adjust the test image in accordance with the location. That is, the test image will be adjusted by the apparatus 300 in accordance with the location of a person, such as the surgeon 402 in the exemplary situation of FIG. 4A, before the test image is projected onto the scene. This enables the features of the scene to be correctly verified regardless of the position of the person viewing the scene.

It will be appreciated that the apparatus 300 may receive the location information from an external device, or alternatively, the apparatus 300 may comprise additional sensors which are used to determine the location of the person viewing the scene. In the case whereby there are a number of persons viewing the scene, a single one of these persons may be identified as the operator and the test image may be adjusted in accordance with the location of the operator.

FIG. 12 illustrates an exemplary situation of the correction of a projection for the operator location according to embodiments of the disclosure. In this example, the test image which has been created by the apparatus 300 in accordance with the initial information received from the machine vision system, and the operator information is projected onto the scene by a projecting unit 1200 under the control of the apparatus 300. In this example, the feature to be verified is the machine vision understanding of the topology of the surface 1202. The operator is intended to view the projection of the test image onto the surface from the predetermined location 1204. If the machine vision system has correctly determined the topology of the surface 1202 then, when viewed from predetermined location 1204, the operator will see that the projection of the test image appears undistorted on the surface, as described with reference to FIG. 8 above.

However, consider that the operator moves from predetermined location 1204 to a new location 1206. In this case, the viewing angle of the projection has changed, while the distance of the operator from the surface has remained constant. However, owing to the change in viewing angle, if the projector 1200 continues to project the same test image onto the surface then the operator may see that the projection is distorted and does not match the comparison image. The operator may therefore incorrectly assume that the machine vision system has misunderstood the topology of the surface. However, the distortion in the projected image actually arises because the operator has changed their viewing location from the predetermined location 1204 and the test image has not yet been adapted accordingly.

That is, it will be appreciated that the operator is seeing the test image reflecting off different portions of the surface depending on their viewing angle and therefore the test image needs to be adapted in accordance with this viewing angle, using the topology information provided by the machine vision system, to take account of the portion of the surface the operator is viewing at any given time.

Once the apparatus 300 has modified the test image in accordance with the change of viewing angle of the operator, then the operator will, if the understanding of the topology of the surface is correct, see an undistorted image of the test image projected onto the scene.

Consider that the operator subsequently changes their location from location 1206 to location 1208. In this case, both the viewing angle and the distance from the surface have changed. Accordingly, if the projection remains constant, then the operator will view a distorted image on the scene and may thus incorrectly assume that the machine vision system has misinterpreted the surface topology. As such, the apparatus 300 needs to take account of the new location from which the operator is viewing the projection in order that the operator can correctly compare whether or not the surface topology has been understood by the machine vision system.

In other words, the test image has to be adapted according to the topology of the portion of the surface the light is being reflected from, with the portion of the surface the light is being reflected from changing in accordance with the viewing angle and viewing distance of the operator from the surface. Otherwise, the wrong portion of the surface topology will be used to correct the test image and a distorted image will be seen by the operator, even if the topology of the surface has actually been correctly determined by the machine vision system.

In contrast, if a distorted image of the test image is seen by the operator even when the test image has been produced by using the initial information and taking account of the operator location, then apparatus 300 can determine that further calibration of the machine vision system which provided the initial information is required.

While the exemplary situation depicted in FIG. 12 has been described with reference to the surface topology, it will be appreciated that the correction for the location of the operator can be applied to any of the features of the scene which are verified using a projection of the test image onto the surface, including the colour and/or brightness of the scene, the translucence of the scene or the like.

It will be appreciated that the manner by which the operator location is determined according to embodiments of the disclosure is not particularly limited. That is, as described above, according to embodiments of the disclosure, the location information may be determined by an external device and provided to the apparatus 300. Alternatively, apparatus 300 may comprises additional sensors which are used to determine the location of the operator relative to the scene.

For example, considering the exemplary situation defined with reference to FIG. 4A, the operator location may be determined by the machine vision system 406. It will be appreciated that the machine vision system 406 used to determine features of the scene may comprise a number of camera systems or the like. These camera systems are primarily used to determine the initial information which is provided to the apparatus 410 for feature verification. However, the camera or camera systems used by the machine vision system 406 to determine the initial information can also be used to determine other features within the operating room 400, provided these features are within the field of view of the machine vision system 406. The operator location information could then be provided to the apparatus 410 by the machine vision system 406 in order that the test image for projection can be correctly produced by the apparatus 410.

Alternatively or in addition, a number of independent camera systems may be used to determine the operator location. In the exemplary situation of FIG. 4A, a single additional ceiling mounted camera system or the like could be provided which captures images of the entire operating room 400. Image processing could then be performed on the image feed from this camera system in order to determine the operator location. The operator location information could then be provided to the apparatus 410 and used, with the initial information from the machine vision system 406, in order to produce the test image for projection on the scene.

Alternatively or in addition, the operator location could be determined using a number of wearable technologies. That is, the operator could be required to wear a small device, such as a band, which provides location information to the apparatus 300 via wireless communication. The location information provided by the wearable technology could be based on GPS, Bluetooth or the like.

Alternatively or in addition, according to embodiments of the disclosure, the apparatus may further be configured to detect the location using indoor location technologies. That is, the location of the operator could be determined using lights, radio waves, magnetic fields, acoustic signals or the like. For example, the location of the operator could be determined using WiFi reflection techniques, where the objects and their location are identified using reflected ambient WiFi signals. Once the location of the operator has been determined in this manner, the location information can be combined with the initial information from the machine vision system by the apparatus 300 in order to produce the test image for projection.

It will be appreciated that these exemplary methods of determining the location of the operator may be used individually, or may alternatively be used in combination in order to provide a more accurate location of the operator to the apparatus 300.

According to embodiments of the disclosure, the apparatus 300 can use the variation in viewing location in order to provide additional levels of certainty when verifying the features of the scene. That is, it will be appreciated that, as described above, when an operator views the test image projected onto the scene they are verifying the feature of the scene for the given portion of the scene which the light they observe is reflected off. In many situations, viewing the scene from a single location may provide a high enough level of certainty that the features of the scene have been correctly identified by the machine vision system. However, in certain situations, the operator may require additional conformation that the feature of the scene has been correctly determined. That is, for certain situations, the operator may wish to test features of the scene from multiple locations in order to provide additional certainty that the features of the scene have been correctly determined.

Consider the example of the topology of the surface. In certain situations, checking that the test image can be projected distortion free onto the surface from a single viewing location (and thus sampling a portion of the topology) may be sufficient in order to verify that the topology of the surface has been correctly determined. However, in more complex situations, or situations where the consequences of a misunderstanding in topology would be severe, then the operator may wish to check that the test image can be projected distortion free onto the surface from multiple viewing locations (thus sampling multiple portions of the topology). Verifying that the test image can be projected distortion free onto the surface when viewed from a number of locations provides an increased level of certainty that the topology has been correctly determined.

According to embodiments of the disclosure, the indication that the feature of the scene should be verified from a number of locations can be provided by the operator by means of an input device, input command or the like. Alternatively, the indication that the feature of the scene should be verified from a number of locations can be provided in the test information which is retrieved by the apparatus 300 in accordance with the operator information. In this case, the test information may indicate the different locations from which the feature of the scene to be verified. The test image may then be projected onto the scene for a number of operator locations in sequence, with the operator asked to compare the projection of the test image for each location in turn. The location from which the operator is intended to view the projection of the test image could, for example, be indicated to the operator using the augmented reality projector or the like.

Comparing the projection of the test image from a number of locations in this manner enables a higher level of confidence to be provided to the user that the feature of the scene has been correctly determined when verifying the feature of the scene according to embodiments of the disclosure.

<Automatic Feature Verification>

In the above described embodiments of the disclosure, the comparison information provided to the apparatus 300 has been produced by an observer who is viewing the overlay of the test image and the scene. Consider the exemplary situation described with reference to FIG. 4A. In this exemplary situation, the surgeon 402 views the scene overlaid with the test image (either on a display, augmented reality glasses, an augmented reality projector or the like) and compares this with the associated predetermined image. The surgeon 402 then provides the apparatus 410 with the comparison information, which the apparatus 410 then uses in order to generate a verification status for that feature. In this manner, the above described embodiments of the disclosure establish an increased sense of trust between the surgeon 402 and the machine vision system 406. That is, because the surgeon 402 can intuitively assess the level of understanding the machine vision system 406 of a robotic device 408 possess regarding a scene, the surgeon 402 can have an increased level of confidence that the robotic device 408 will perform an assigned task correctly without any misunderstanding of the features of the scene.

However, there are situations whereby the operator may not be present, and may thus be unable, to provide the comparison information to the apparatus 300 in this manner. Alternatively, the operator may be present but, owing to other external pressures and demands, be unable to provide the comparison information at that time. In this situation, according to embodiments of the disclosure, the comparison information may be produced by the apparatus 300 itself. According to embodiments of the disclosure, the comparison information includes a result of machine vision of the at least one test image overlaid with the scene, the machine vision being performed on sensor information generated by a machine vision system. That is, the machine vision system will capture sensor information (such as an image of the at least one test image overlaid with the scene) and will perform machine vision analysis on the sensor information in order to produce comparison information of the at least one test image overlaid with the scene and the at least one predetermined image.

Furthermore, in embodiments of the disclosure, the apparatus 300 may be further configured to receive an image of the at least one test image overlaid with the scene; produce comparison information relating to the comparison of the image of at least one test image overlaid with the scene with the at least one predetermined image selected in accordance with the test information and generate a verification status of a feature of the scene in accordance with the comparison information which has been produced.

It will be appreciated that the production of the comparison information in this manner requires the projection of the test image onto the surface using an augmented reality projector or the like.

In other words, the apparatus 300 according to embodiments of the disclosure projects the test image onto the scene and then, using an independent camera system or the like, captures an image of the test image as it appears when projected onto the scene. The apparatus 300 is then configured to perform the comparison between the image of the projection of the test image and the associated predetermined image in order to produce comparison information for that test image. The apparatus 300 will then generate the verification status of the corresponding feature of the scene in the same manner as described above with reference to the embodiments whereby the comparison information has been produced by a human operator.

Consider the exemplary situation described with reference to FIG. 4A. In this example, the surgeon 402 has requested that the machine vision system 406 understanding of the topology of the scene is verified by the verification apparatus 410. That is, the surgeon 402 has provided information regarding the operation to be performed, and the apparatus 410 has determined from the corresponding test information retrieved on the basis of this information that a feature of the machine vision system 406 understanding to be verified before preforming the operation is the topology of the surface of the surgical site. According to embodiments of the disclosure, the apparatus 410 produces a test image using a predetermined image selected in accordance with the feature to be verified and the initial information of the scene. The apparatus 410 then projects this image onto the scene. For example, in certain embodiments the apparatus 410 further comprises a projector, such as an augmented reality projector or the like, which will project the image onto the surface. The projection of the image onto the scene may highlight certain portions of the scene; this is described in more detail with reference to the exemplary methods FIGS. 8 to 11 above.

According to embodiments of the disclosure, once the test image has been projected onto the scene in this manner, the apparatus 410 receives an image of the scene with the test image projected onto it. That is, in the exemplary situation described with reference to FIG. 4A for example, an additional external camera system located in the surgery 400 will capture an image of the scene with the test image projected onto it, and will provide the image to the apparatus 410. Of course, it will be appreciated, as described above with reference to human observer, the additional camera system will have to capture an image of the scene from a predetermined location within the surgical theatre 400. Alternatively, the additional camera system could provide the apparatus 410 with its location information, and the apparatus 410 could adjust the test image for projection accordingly. Further alternatively, the additional camera system could be a camera provided as part of the apparatus 410 itself, and the apparatus 410 will capture the image from its own location. Regardless, according to embodiments of the disclosure, the apparatus 410 receives an image of the projection of the test image onto the scene.

Once the apparatus 410 has received the image of the projection of the test image onto the scene, the apparatus 410 is configured to perform a comparison of this image with the associated predetermined image. If the apparatus 410 has determined that the machine vision system 406 understanding of surface topology needs to be verified, then the predetermined image may be a grid similar to 800, the test image may be a distorted grid similar to 804 and the image of the test image projected onto the scene may be an image similar to image 808 described with reference to FIG. 8. Upon receiving the image of the test image projected onto the scene, apparatus 410 may then perform a comparison between that image and the predetermined image. That is, in this example, the apparatus 410 may determine whether the test image projected onto the scene appears distorted, or whether, when projected onto the scene, the test image appears the same as the original predetermined image.

Furthermore, the comparison between these images may be based on a threshold level for example. That is, if the apparatus 410 determines that the match between the image of the test image projected onto the scene and the predetermined image is too low (that is, there is a large amount of distortion still present in the image of the projected test image) then the apparatus 410 will determine that the corresponding feature, which in this exemplary situation is the topology, has not been satisfactorily determined and therefore should not be verified.

It will be appreciated that the threshold level of similarity required may vary depending on the situation. For example, in some embodiments, the threshold level of similarity required may be indicated by the test information which is retrieved by the apparatus 410 using the operator information. In certain situations, the test information may indicate that a detailed understanding of the topology is not required, while a detailed understanding of the colour variation in the image is required. In this case, the threshold level of similarity required in the comparison of the image of the test image projected on the scene and the predetermined image may be set lower when assessing the understanding of topology than when assessing the understanding of the colour variation.

It will be appreciated that the method by which the apparatus 300 according to embodiments of the disclosure performs the image comparison is not particularly limited. For example, a pixel based comparison, a block based comparison, a histogram based comparison, a feature-based comparison or the like may be used. Of course, a combination of these techniques may be used to provide a combined indication of the degree of similarity between the images which can be compared with the threshold level of similar for that feature. The actual method used by the apparatus 300 will depend upon the context of the situation in which embodiments of the disclosure are implemented.

Furthermore, it will be appreciated that, according to embodiments of the disclosure, the automatic production of the comparison information may be used in combination with the comparison information provided by the human operator. That is, the apparatus 300 may be configured to combine the comparison information provided by the human operator with the comparison information determined by the apparatus 300 itself in order to generate the verification status of the feature. In embodiments, the two sources of comparison information could have equal weighting in the generation of the verification status. Alternatively, the human comparison information could take precedence over the comparison information provided by the apparatus 300 itself, with the comparison information provided by the apparatus 300 being used as a safety check on the comparison information provided by the human operator.

For example, if the comparison information provided by the human operator appears to indicate that there is good comparison between the projection of the test image on the scene and the associated predetermined image, yet the comparison information produced by the apparatus 300 indicates that the comparison between these two images is poor, then the apparatus 300 may alert the human operator to the discrepancy. Upon receiving notification of this discrepancy, the human operator may further review the test image and can decide whether or not they wish to update their comparison information. If the human operator confirms their original comparison information, then the apparatus 300 will proceed to generate the verification information in accordance with the human comparison information alone. However, if the human operator instead decides to revise the comparison information, then the apparatus 300 will produce the verification status on the basis of this revised comparison information.

Such a discrepancy between the human comparison information and the comparison information produced by the apparatus 300 may occur for a number of reasons. For example, the human operator may have been partially distracted when providing the comparison information, or alternatively, may have provided the comparison information in error. Regardless of the source of the discrepancy in the comparison information, combining the comparison information of the human operator and the apparatus 300 in this manner further improves the verification of the features of the scene according to embodiments of the disclosure thus leading to a reduction in the misinterpretation of features of the scene by a machine vision system.

<Additional Modifications>

It will be appreciated that while embodiments of the disclosure have been described with reference to verification of machine vision systems for robotic systems in surgery, the present disclosure is not intended to be limited in this regard. That is, the apparatus, system and methods for verifying features of the scene according to embodiments of the disclosure may alternatively be applied to any number of exemplary situations where features of a scene determined by machine vision systems or the like require external verification. For example, within medical situations, embodiments of the disclosure may be applied to endoscopic surgery systems or the like. Furthermore, embodiments of the disclosure can be applied outside medical situations, and may alternatively be used, for example, to verify the machine vision systems of other autonomous or semi autonomous robotic devices including fault recognition systems, vehicles navigation systems or the like.

Various embodiments of the present disclosure are defined by the following numbered clauses:

(1)
A verification system for verifying features of a scene, the system including: circuitry configured to:
receive initial information determined in accordance with a first analysis of the scene;
produce at least one test image in accordance with test information indicating at least one feature of the scene to be verified, the at least one test image being at least one predetermined image selected in accordance with the test information, modified in accordance with the initial information;
overlay the scene with the at least one test image;
receive comparison information relating to a comparison of the at least one test image overlaid with the scene with the at least one predetermined image selected in accordance with the test information; and
generate a verification status of a feature of the scene in accordance with the received comparison information.

(2)
The system according to Clause 1, wherein the initial information includes detection or recognition information from a sensor information generated by a machine vision system.

(3)
The system according to any preceding Clause, wherein the comparison information includes a result of machine vision of the at least one test image overlaid with the scene, the machine vision being performed on sensor information generated by a machine vision system.

(4)
The system according to any preceding Clause, wherein the test information is retrieved from a storage unit, in accordance with operator information.

(5)
The system according to any preceding Clause, wherein the test information further indicates a required accuracy level of feature verification and the circuitry is further configured to produce the at least one test image in accordance with this accuracy level requirement.

(6)
The system according to any preceding Clause, wherein when the at least one test image includes a plurality of test images, the circuitry is further configured to overlay the scene with the at least one test image in a sequence, and is further configured to receive comparison information for each of the test images in turn.

(7)
The system according to any preceding Clause, wherein the circuitry is further configured to overlay the at least one test image in order that features of the scene are highlighted in accordance with the initial information.

(8)
The system according to any preceding Clause, wherein the circuitry is further configured to produce the test image in accordance with information regarding the operating environment.

(9)
The system according to any preceding Clause, wherein the circuitry is further configured to receive the comparison information using speech recognition.

(10)
The system according to any preceding Clause, wherein the circuitry is further configured to generate comparison questions in accordance with the test information.

(11)
The system according to any preceding Clause, wherein the circuitry is further configured to:
receive an image of the at least one test image overlaid with the scene;
produce comparison information relating to the comparison of the image of at least one test image overlaid with the scene with the at least one predetermined image selected in accordance with the test information; and
generate a verification status of a feature of the scene in accordance with the comparison information which has been produced.

(12)
The system according to any preceding Clause, wherein the circuitry is further configured to request adjustment of the initial information when the verification status of the feature of the scene indicates that the feature could not be verified.

(13)
The system according to any preceding Clause, wherein overlaying the scene with the at least one test image comprises displaying the at least one test image on a display.

(14)
The system according to any preceding Clause, wherein overlaying the scene with the at least one test image comprises projecting the at least one test image onto the scene.

(15)
The system according to Clause 7 wherein the circuitry is further configured to detect a location of a person viewing the projection and adjust the test image in accordance with the location.

(16)
The system according to Clause 15, wherein the circuitry is further configured to detect the location using indoor location technologies.

(17)
The system according to Clause 11, wherein the system further including an projection apparatus configured to project the at least one test image to be overlaid on the scene, in order to verify at least one feature of the scene including the topology of the scene, the colour variation of the scene, the reflectivity of the scene, the translucence of the scene and brightness variance across the scene.

(18)
The system according to Clause 17, wherein the circuitry is further configured to detect a location of the projection apparatus and adjust the test image in accordance with the location.

(19)
A verification method of verifying features of a scene, the method including:
receiving initial information determined in accordance with a first analysis of the scene;
producing at least one test image in accordance with test information indicating at least one feature of the scene to be verified, the at least one test image being at least one predetermined image selected in accordance with the test information, modified in accordance with the initial information;
overlaying the scene with the at least one test image;
receiving comparison information relating to a comparison of the at least one test image overlaid with the scene with the at least one predetermined image selected in accordance with the test information; and
generating a verification status of a feature of the scene in accordance with the received comparison information.

(20)
A computer program product comprising instructions which, when the program is executed by a computer, cause the computer to carry out the method including:
receiving initial information determined in accordance with a first analysis of the scene;
producing at least one test image in accordance with test information indicating at least one feature of the scene to be verified, the at least one test image being at least one predetermined image selected in accordance with the test information, modified in accordance with the initial information;
overlaying the scene with the at least one test image;
receiving comparison information relating to a comparison of the at least one test image overlaid with the scene with the at least one predetermined image selected in accordance with the test information; and
generating a verification status of a feature of the scene in accordance with the received comparison information.

Obviously, numerous modifications and variations of the present disclosure are possible in light of the above teachings. It is therefore to be understood that within the scope of the appended claims, the disclosure may be practiced otherwise than as specifically described herein.

In so far as embodiments of the disclosure have been described as being implemented, at least in part, by software-controlled data processing apparatus, it will be appreciated that a non-transitory machine-readable medium carrying such software, such as an optical disk, a magnetic disk, semiconductor memory or the like, is also considered to represent an embodiment of the present disclosure.

It will be appreciated that the above description for clarity has described embodiments with reference to different functional units, circuitry and/or processors. However, it will be apparent that any suitable distribution of functionality between different functional units, circuitry and/or processors may be used without detracting from the embodiments.

Described embodiments may be implemented in any suitable form including hardware, software, firmware or any combination of these. Described embodiments may optionally be implemented at least partly as computer software running on one or more data processors and/or digital signal processors. The elements and components of any embodiment may be physically, functionally and logically implemented in any suitable way. Indeed the functionality may be implemented in a single unit, in a plurality of units or as part of other functional units. As such, the disclosed embodiments may be implemented in a single unit or may be physically and functionally distributed between different units, circuitry and/or processors.

Although the present disclosure has been described in connection with some embodiments, it is not intended to be limited to the specific form set forth herein. Additionally, although a feature may appear to be described in connection with particular embodiments, one skilled in the art would recognize that various features of the described embodiments may be combined in any manner suitable to implement the technique.

1. A verification system for verifying features of a scene, the system comprising:
circuitry configured to:
receive initial information determined in accordance with a first analysis of the scene;
produce at least one test image in accordance with test information indicating at least one feature of the scene to be verified, the at least one test image being at least one predetermined image selected in accordance with the test information, modified in accordance with the initial information;
overlay the scene with the at least one test image;
receive comparison information relating to a comparison of the at least one test image overlaid with the scene with the at least one predetermined image selected in accordance with the test information; and
generate a verification status of a feature of the scene in accordance with the received comparison information.

2. The system according to claim 1, wherein the initial information includes detection or recognition information from a sensor information generated by a machine vision system.

3. The system according to claim 1, wherein the comparison information includes a result of machine vision of the at least one test image overlaid with the scene, the machine vision being performed on sensor information generated by a machine vision system.

4. The system according to claim 1, wherein the test information is retrieved from a storage unit, in accordance with operator information.

5. The system according to claim 1, wherein the test information further indicates a required accuracy level of feature verification and the circuitry is further configured to produce the at least one test image in accordance with this accuracy level requirement.

6. The system according to claim 1, wherein when the at least one test image comprises a plurality of test images, the circuitry is further configured to overlay the scene with the at least one test image in a sequence, and is further configured to receive comparison information for each of the test images in turn.

7. The system according to claim 1, wherein the circuitry is further configured to overlay the at least one test image in order that features of the scene are highlighted in accordance with the initial information.

8. The system according to claim 1, wherein the circuitry is further configured to produce the test image in accordance with information regarding the operating environment.

9. The system according to claim 1, wherein the circuitry is further configured to receive the comparison information using speech recognition.

10. The system according to claim 1, wherein the circuitry is further configured to generate comparison questions in accordance with the test information.

11. The system according to claim 1, wherein the circuitry is further configured to:
receive an image of the at least one test image overlaid with the scene;
produce comparison information relating to the comparison of the image of at least one test image overlaid with the scene with the at least one predetermined image selected in accordance with the test information; and
generate a verification status of a feature of the scene in accordance with the comparison information which has been produced.

12. The system according to claim 1, wherein the circuitry is further configured to request adjustment of the initial information when the verification status of the feature of the scene indicates that the feature could not be verified.

13. The system according to claim 1, wherein overlaying the scene with the at least one test image comprises displaying the at least one test image on a display.

14. The system according to claim 1, wherein overlaying the scene with the at least one test image comprises projecting the at least one test image onto the scene.

15. The system according to claim 7 wherein the circuitry is further configured to detect a location of a person viewing the projection and adjust the test image in accordance with the location.

16. The system according to claim 15, wherein the circuitry is further configured to detect the location using indoor location technologies.

17. The system according to claim 11, wherein the system further comprising an projection apparatus configured to project the at least one test image to be overlaid on the scene, in order to verify at least one feature of the scene including the topology of the scene, the colour variation of the scene, the reflectivity of the scene, the translucence of the scene and brightness variance across the scene.

18. The system according to claim 17, wherein the circuitry is further configured to detect a location of the projection apparatus and adjust the test image in accordance with the location.

19. A verification method of verifying features of a scene, the method comprising:
receiving initial information determined in accordance with a first analysis of the scene;
producing at least one test image in accordance with test information indicating at least one feature of the scene to be verified, the at least one test image being at least one predetermined image selected in accordance with the test information, modified in accordance with the initial information;
overlaying the scene with the at least one test image;
receiving comparison information relating to a comparison of the at least one test image overlaid with the scene with the at least one predetermined image selected in accordance with the test information; and
generating a verification status of a feature of the scene in accordance with the received comparison information.

20. A computer program product comprising instructions which, when the program is executed by a computer, cause the computer to carry out the method comprising:
receiving initial information determined in accordance with a first analysis of the scene;
producing at least one test image in accordance with test information indicating at least one feature of the scene to be verified, the at least one test image being at least one predetermined image selected in accordance with the test information, modified in accordance with the initial information;
overlaying the scene with the at least one test image;
receiving comparison information relating to a comparison of the at least one test image overlaid with the scene with the at least one predetermined image selected in accordance with the test information; and
generating a verification status of a feature of the scene in accordance with the received comparison information.
